(19) United States
(12) Patent Application Publication
(10) Pub. No.: US 2006/0159722 A1
(43) Pub. Date: Jul. 20, 2006

(54) Layered aligned polymer structures and methods of making same

(76) Inventors: Gavin J C Braithwaite, Cambridge, MA (US); Jeffrey W Ruberti, Lexington, MA (US)

Correspondence Address: HELLER EHRMAN WHITE & MCAULIFFE LLP, 1717 RHODE ISLAND AVE, NW, WASHINGTON, DC, 20036-3001, US

(21) Appl. No.: 10/536,409

(22) PCT Filed: Nov. 26, 2003
(86) PCT No.: PCT/US03/38087

§ 371 Date: Jan. 23, 2006

Related U.S. Application Data
(63) Continuation-in-part of application No. 10/306,825, filed on Nov. 27, 2002, now Pat. No. 7,048,963.
Application No. PCT/US03/38087 is a continuation-in-part of application No. 10/611,674, filed on Jun. 30, 2003.
(60) Provisional application No. 60/337,286, filed on Nov. 30, 2001.

(51) Int. Cl. C12N 5/08; A61K 9/70; A61K 35/44

(52) U.S. Cl. 424/427; 424/443; 435/368

(57) ABSTRACT

This invention includes a method of producing a nanostructured artificial template comprising one or more thin, oriented layer of polymer material. The material is preferably produced by the method of introducing a shearing flow in a predominantly monomeric solution of the self-assembling polymer sub-units to the free surface of a substrate and inducing polymerization or growth of the monomer while in this shearing flow. The rate of flow of the material from the delivery system and the relative velocity between the deposition surface and the material as it is delivered to the surface are controlled to properly orient the material at the desired thickness. These rates can be adjusted to vary the properties of the film in a controlled manner. The nanostructured artificial template is useful for inducing the production of a templated extracellular matrix by a population of cells. The invention further includes a method of remodeling collagen constructs by alternating application of proteases and collagen monomers while the construct is stressed.

### CROSS REFERENCES TO RELATED APPLICATIONS

This application is a continuation-in-part of co-pending U.S. application Ser. No. 10/611,674, filed on Jun. 30, 2003 which is a continuation-in-part of U.S. application Ser. No. 10/306,825, filed on Nov. 27, 2002, which claims benefit of U.S. Provisional Application No. 60/337,286 filed on Nov. 30, 2001, the entire contents of which are incorporated herein by reference in their entirety.

### BACKGROUND OF THE INVENTION

Accurate control of the orientation of polymeric structure in thin layers is desired to maximize their mechanical, chemical, and optical properties. While orientation can be performed through mechanical means, it is often more desirable to orient the structure during the polymerization process, particularly if the process involves polymerizing the system into a specific final shape, where further mechanical manipulation is unfeasible.

Biomedical components often require oriented structures.. Tendons, for example, contain highly oriented collagen fibrils, and the spinal intervertebral disc is composed mainly of oriented crystalline collagen fibrils and amorphous hydrophilic proteoglycan. The prevalence of oriented collagen in the human body makes formation of highly oriented layers of this polymer a desired goal in modeling of tissue structure. Collagen is a biopolymer and protein, and found in the structure of tendons, skin, bones and blood vessels. Randomly oriented collagen materials are weak, and degrade quickly when exposed to mechanical stress. Soluble collagen is derived from animal tissue, and can be obtained in a monomeric form. The collagen monomer will polymerize, termed “fibrillogenesis,” to form a gel-like structure. This process is achieved when the collagen is in a solution of specific pH temperature, and ionic strength. The polymerization occurs as a self-assembly process, producing native-type collagen fibers. The fibers grow into a porous gel matrix.

The collagen molecule is a rod-like structure in the unaggregated state, composed of three peptide chains intertwined to form a triple helix. Improvements over randomly oriented gels in mechanical and optical properties can be realized if the aggregated molecules can be coerced into an oriented, or aligned, state. Cast films of collagen can form a randomly oriented gel structure, which lacks desired mechanical and optical properties. It is desirable to give a structured order for optical transparency in corneal replacements.

Collagen fiber orientation techniques have included mechanical deformation of already gelled matrices and laminar flow of a polymerizing matrix. These approaches yield gelled layers hundreds of microns thick.

### SUMMARY OF THE INVENTION

The present invention provides methods of making a templated extracellular matrix that are useful for making structured connective tissues such as a full thickness corneal stroma. In a preferred embodiment, the invention provides a method of producing a structured connective tissue comprising the steps of providing a nanostructured artificial template (NAT), contacting the NAT with a population of fibroblastic cells to produce a templated extracellular matrix, and stacking a plurality of templated extracellular matrices. The template in accordance with a preferred embodiment is generated using shear flow. In preferred embodiments, the structured connective tissue is corneal stroma, ligament, tendon, fascia or annulus fibrosis.

In other preferred embodiments, the invention provides methods of producing a templated extracellular matrix, comprising the steps of controlling a flow of a predominantly monomeric polymer solution onto a substrate, wherein the resulting shear flow produces aligned polymer structures; controlling a plurality of parameters during polymerization; generating a first layer of nanostructured artificial template; contacting the first layer of nanostructured artificial template with a first population of cells; and maintaining the nanostructured artificial template and the first population of cells in a culture to produce a templated extracellular matrix. In preferred embodiments, the method further comprises the step of generating at least one additional layer, wherein each successive layer comprises a portion of the templated extracellular matrix.

In another preferred embodiment, the invention provides a method of producing a templated extracellular matrix, comprising the steps of providing a nanostructured artificial template; contacting the nanostructured artificial template with a first population of cells; and maintaining the nanostructured artificial template and the first population of cells in culture to produce a templated extracellular matrix having a first surface and a second surface.

In preferred embodiments, the polymer structures are self-assembling collagen fibrils. In preferred embodiments, the solution comprises collagen monomers. In embodiments wherein the solution comprises collagen monomers, the solution preferably comprises a phosphate buffered saline solution having a pH in the range of about 7.2 to about 7.6.

In embodiments in which the templated extracellular matrix comprises additional successive layers, the layers typically are about 0.1 micrometer to about 100 micrometer thick. In embodiments comprising collagen, the collagen is type I collagen, type V collagen or mixtures thereof. The collagen fibrils produced can be homotypic or heterotypic fibrils.

In preferred embodiments, the polymer structures of each layer have a preferential alignment. The preferential alignment in a single layer typically varies among the successive layers. In general, the angle between the preferential orientation of the successive layers is in the range of 0 to 180 degrees.

Modulation of controlled parameters can determine the polymerization kinetics and the polymer structures formed. Typically the controlled parameters include solution properties such as temperature, monomer concentration and the amount and type of surfactant present. Other controlled parameters can include the temperature and relative humidity of the ambient air. In embodiments in which the polymer is a biopolymer such as collagen, the monomer concentration is in the range of 0.01 to 100 mg/ml, preferable 0.5 to 10 mg/ml. In a preferred embodiment, the predominantly monomeric solution includes about 3 mg/ml Type I collagen.

In certain embodiment, the shear flow is generated by spinning the substrate at a controlled rate of about 50 Hz to about 50,000 Hz. In other embodiments, the shear flow is generated by spinning the substrate at about 10 Hz to about 10,000 Hz. In another embodiment, the substrate is spun at about 250 rpm to about 3200 rpm. In further embodiments, the shear flow is generated by drawing the substrate out of the monomer solution.

In preferred embodiments, the parameters of solution flow rate, solution viscosity and substrate rotational velocity are controlled. Typically, the parameters of solution flow rate, solution viscosity and substrate rotational velocity are controlled to produce a shear rate between 1 s⁻¹ and 500,000 s⁻¹, preferably between 10 s⁻¹ to 10,000 s⁻¹. In some embodiments, the solution flow is controlled at a constant rate of about 0.05 to about 1,000 ml/min, preferably between about 0.1 to about 100 ml/min. In other embodiments, the solution viscosity is controlled in the range of about 1 mpascal-sec. In one embodiment, the viscosity is about 10 mpascal-sec.

The predominantly monomeric solution is suitably aqueous or non-aqueous, depending on the monomer. In preferred embodiments, the pH of the solution is controlled. In specific preferred embodiments, the solution is an aqueous solution, and the pH is buffered, preferably in the range of about 7.2 to about 7.6. Any buffer capable of maintaining the pH within this range is suitable; a preferred buffer is a phosphate buffer.

The predominantly monomeric solution can include one or more additives besides the monomers. Additives can be chosen to roles such as promoting polymerization, combining with the monomers to form a copolymer, or providing a coating on the polymer structures. In embodiments in which the polymer is collagen, a preferred additive is one or more glycosaminoglycans selected from the group consisting of hyaluronan, chondroitin sulfate, dermatan sulfate, keratin sulfate, or proteoglycans selected from the group including decorin, lumican, biglycan, keratocan, syndican and mixtures thereof Additives can be chosen to modify the physical properties of the monomer solution. For example, glycerol can be added to adjust the viscosity of the monomer solution. In other embodiments, a surfactant can be added to improve wetting of the substrate.

In other preferred embodiments, the invention provides a biomimetic corneal stroma produced by the steps of providing a nanostructured artificial template; contacting the nanostructured artificial template with a first population of eukaryotic cells; maintaining the nanostructured artificial template and the first population of cell in culture to produce a templated extracellular matrix; repeating the steps of providing, contacting and maintaining to produce additional templated extracellular matrices; and stacking a plurality of templated extacellular matrices oriented at any arbitrary angle with respect to each other. Typically the eukaryotic cells are mammalian fibroblasts, preferably human keratocytes. In preferred embodiments, the eukaryotic cells are treated with an ascorbate compound to activate secretion of extracellular matrix. The eukaryotic cells can be activated before or after coming into contact with NAT. In preferred embodiments, activated cells are made quiescent by removal of the ascorbate compound and preferably additionally contacting the cells with fetal bovine serum. In preferred embodiments, the biomimetic corneal stroma is transparent.

In a preferred embodiment, the present invention provides a biomimetic cornea produced by the steps of providing a nanostructured artificial template; contacting the nanostructured artificial template with a first population of eukaryotic cell; maintaining the nanostructured artificial template with the eukaryotic cells to form a template extracellular matrix; repeating the steps of providing, contacting and maintaining to form additional templated extracellular matrices; stacking a plurality of templated extracellular matrices oriented at any arbitrary angle with respect to one another to form a multilaminar templated extracellular matrix; contacting a first surface of the multilaminar templated extracellular matrix with a second population of cells; and maintaining the multilaminar templated extracellular matrix in culture to form a biomimetic cornea. In preferred embodiments, the biomimetic cornea is transparent.

In other embodiments, the invention provides a method of producing a nanostructured artificial template comprising one or more thin, oriented layers of polymer material. The material is preferably produced by the method of introducing a shearing flow to a free surface in a predominantly monomeric solution of the self-assembling polymer sub-units, and inducing polymerization or growth of the monomer while in this shearing flow. The system for forming the oriented layer of material provides shear flow between a delivery system and the substrate on or over which the material is deposited. In general, any method to produce shear, elongation, or parabolic flows between substrates may be used. The rate of flow of the material from the delivery system, the relative velocity between the deposition surface and the material as it is delivered to the surface, or both, can be controlled to properly orient the material at the desired thickness. These rates can be adjusted to vary the properties of the film in a controlled manner. Preferred embodiments include either angular or linear flow between the delivery system and the substrate.

In many embodiments the predominantly monomeric solution comprising monomers is deposited on a flat substrate. Alternatively, deposition of the solution can occur on a substrate having curved or spherical surfaces to result in stress-free interlayer boundaries. Such curved embodiments are useful for corneal constructs.

In a preferred embodiment, the step of controlling the temperature, pH, solvent chemistry, and relative humidity during the polymerzation process is performed on a local level within the adjacent environment where the polymerization occurs.

The preferred embodiment of the present invention further comprises a layered construct composed of layers of about 0.1 μm to about 100 μm thick oriented polymeric films or fibers, with the principal direction of orientation differing between each subsequent layer. A preferred embodiment of the invention uses this method to form synthetic biocompatible or biopolymeric materials such as implantable tissue material. These materials can be implanted as soft tissue replacement or for bone or joint replacement or repair.

In embodiments in which the biopolymer is collagen, the method further includes inducing fibrillogenesis of the collagen while in the shearing flow. The method further includes controlling the collagen monomer concentration, temperature, solution properties and relative humidity of the fibrillogenesis process, producing collagen material having an oriented fibrillar structure in a sheet with a uniform, controllable thickness. The thickness can range from about 0.5 μm to about 100 μm.

In accordance with another preferred embodiment, the method for producing a multi-layer construct can be used to form an artificial corneal construct. In preferred embodiments, the artificial corneal construct is transparent. The collagen layers can be seeded with endothelial and epithelial cells, which generate a negative pressure field in the construct, and compress the construct to a thickness necessary for optical transparency.

A preferred embodiment includes a method of producing a thin film of oriented polymer structures, including the steps of controlling the flow of a predominantly monomeric solution into a device having a substrate, the device generating a shear flow to induce alignment of polymer structures, controlling a plurality of parameters during polymerization; and generating a layer of oriented polymer. The method further includes the polymer being a biopolymer such as collagen The method further comprises the steps of mixing a solution of collagen with phosphate buffered saline solution, adjusting the pH of the solution to the range of about 7.2 to about 7.6, applying the solution at a controlled rate onto a substrate which generates a shearing flow, causing preferential orientation of the gelling collagen fibrils; and generating successive layers, each layer representing a portion of the component. The layers have a uniform, controllable thickness ranging from 0.1 micrometer to 100 micrometers. The collagen can be either type I, type V collagen or mixture thereof The principal orientation of the aligned fibrils in a single layer alternates in each successive layer. The angle between the principal orientation of each successive layer in the range of 0 to <90 degrees. The solution properties, including temperature, concentration and surfactant composition are controlled. The shear flow is generated by spinning the substrate at a controlled rate in a range of approximately 50 to 50,000 Hz.

In other embodiments, the present invention provides a method of making a multilaminar nanostructured template comprising introducing a monomer solution from a first inlet, between a polymer accepting surface and a polymer rejecting surface to first outlet to produce an aligned polymer layer, increasing the spacing between the polymer accepting surface and the polymer rejecting surface; introducing the monomer solution into a second inlet and recovering the monomer solution from a second outlet wherein the flow from the second inlet to the second outlet is substantially orthogonal to the flow from the first inlet to the first outlet; and producing an aligned polymer layer in which the polymer molecules are substantially orthogonal to the polymer molecules of the previous layer.

In a preferred embodiment the present invention provides a method to generate single or multiple layers of aligned polymer fibrils by pumping monomer solution between two surfaces, with an adjustable gap between them. One of the surfaces is collagen accepting and the other surface is collagen rejecting. Single or multiple layers of collagen may be produced by creating collagen self assembly conditions within the gap between the surface. A collagen accepting surface may be generated by coating the surface may be generated by coating the surface with antibodies to collagen, by plasma cleaning, by cleaning with a detergent such as Micro90™, by functionalization, or by treating the surface with any methods known to attract and promote adherence of collagen monomer or polymer units. A collagen rejecting surface may be generated by functionalization, surface treatments, coatings or use of materials known to limit, reduce, or reject the adhesion of collagen monomer or polymer units.

This invention further provides a method of producing a nanostructured artificial template that is useful for directing the production of extracellular matrix by eukaryotic cells such as fibroblasts. In general, the nanostructured artificial template is an aligned array of linear structures sufficient to induce a fibroblast to produce an aligned extracellular matrix which can in turn iteratively act to induce fibroblasts to produce subsequent aligned extracellular matrix. In preferred embodiments, the aggregate extracellular matrix product of the population of fibroblasts is an aligned nanostructured array that functions as a template for further extracellular matrix production by fibroblasts. In preferred embodiments, the aligned extracellular matrix array produced by the population of fibroblasts in response to the nanostructured artificial template is oriented with respect to the linear structures of the nanostructured artificial template. In preferred embodiments, each successive aligned extracellular matrix array are rotated some angle, theta, with respect to the orientation of the previous template, where theta has a value from about zero to about 90 degrees.

In one preferred embodiment, the invention provides products and methods for making an oriented collagenous structure, including the steps of providing a collagen construct; loading the collagen construct; contacting the collagen construct with a solution comprising at least one matrix metalloproteinase; and contacting the collagen construct with a solution comprising collagen monomers. In some preferred embodiments, the method also includes the step of adjusting the load on the collagen construct. In some preferred embodiments, the method also includes the step of contacting the collagen construct with a population of cells. If desired, the step of contacting the collagen construct with a solution comprising at least one matrix metalloproteinase can be repeated one or more times. If desired the step of contacting the collagen construct with a solution comprising collagen monomers can be repeated one or more times.

In certain embodiments, the method includes the step of contacting the collagen construct with a solution including a glycosaminoglycan or proteoglycan. Where the solution comprises a glycosaminoglycan, the glycosaminoglycan can be selected from the group consisting of hyaluronan, chondroitin sulfate, dermatan sulfate, keratan sulfate, heparin, heparin sulfate, and mixtures thereof. Where the solution comprises a proteoglycan, the proteoglycan can be selected from the group consisting of decorin, lumican, biglycan, keratocan, syndican, aggrecan, perlecan, asporin, fibromodulin, epiphycan, PG-Lb, dermatan sulfate proteoglycan-3, versican, mimecan and mixtures thereof In other embodiments, the method includes the step of contacting the collagen construct with a solution comprising a protein selected from the group consisting of collagen type IV, laminin, fibronectin, vinculin, an integrin moiety, and mixtures thereof. The collagen construct can be loaded with a static stress or a dynamic stress. Typically, the collagen construct is loaded with a stress sufficient to produce about 0.1% to about 20% strain. In preferred embodiments, the collagen construct is loaded with a stress of about 0.01 to about 10 MPa. The applied stress can be oriented along a single axis (unaxial stress), or oriented along two axes (biaxial stress). In other embodiments, the collagen construct is loaded with a tangential stress. In further embodiments, the collagen construct is loaded with a three-dimensional stress.

The collagen construct can be an unoriented collagen gel, a tissue-derived collagen template or a nanostructured artificial template. Typically, the collagen construct comprises a collagen is selected from the group consisting of Type I collagen, Type V collagen, and mixtures thereof In preferred embodiments, the collagen construct comprises Type I collagen. In other embodiments, the collagen construct comprises a mixture of Type I collagen and Type V collagen. Preferably, the construct comprises more Type I collagen than Type V collagen. In one preferred embodiment, the construct comprises a mixture of about four parts Type I collagen to about one part Type V collagen. In some embodiments, the collagen construct can also include a collagen that is selected from the group consisting of Type II collagen, Type III collagen, Type XI collagen, Type IV collagen, and mixtures thereof. The self-assembled collagen fibrils can be homotypic or heterotypic.

The matrix metalloproteinase solution includes at least one matrix metalloproteinase selected from the group consisting of MMP-1 (interstitial collagenase, EC 3.4.24.7), MMP-2 (gelatinase-A, EC 3.4.24.24), MMP-3 (stromelysin-1, transin, EC 3.4.24.17), M-7 (matrilysin-1, EC 3.4.24.23), MMP-8 (neutrophil collagenase, collagenase-2, EC 3.4.24.34), MMP-9 (gelatinase-B, EC 3.4.24.35), MMP-10, MMP-11 (stromelysin-3), MMP-12 (metalloelastase macrophage elastase, EC 3.4.24.65), MMP-13 (collagenase-3, EC 3.4.24.-), MMP-18, recombinant catalytic domain fragments thereof and mixtures thereof. Typically, suitable reaction conditions for each enzyme are controlled. The particular matrix metalloproteinase selected depends on the specific type(s) of collagen that form the construct.

Alternatively, the collagen structure can be subjected to a stress by loading the collagen construct internally with at least one hydrophilic moiety that produces swelling pressure by the uptake of water. In such embodiments, the method includes the steps of providing a collagen construct; loading the collagen construct internally by adding at least one retained hydrophilic moiety that increases the swelling pressure of the collagen construct; contacting the collagen construct with a solution comprising at least one matrix metalloproteinase; and contacting the collagen construct with a solution comprising collagen monomers. In some embodiments, the method also includes the step of adjusting the load on the collagen construct. In some embodiments, the method also includes the step of contacting the collagen construct with a population of cells. If desired, the step of contacting the collagen construct with a solution comprising at least one matrix metalloproteinase can be repeated one or more times. If desired the step of contacting the collagen construct with a solution comprising collagen monomers can be repeated one or more times.

In certain embodiments, the retained hydrophilic moiety is selected from the group consisting of glycosaminoglycans, proteoglycans and mixtures thereof. Where the retained hydrophilic moiety is a glycosaminoglycan, the glycosaminoglycan can be selected from the group consisting of hyaluronan, chondroitin sulfate, dermatan sulfate, keratan sulfate, heparin, heparin sulfate, and mixtures thereof Where the retained hydrophilic moiety is a proteoglycan, the proteoglycan can be selected from the group consisting of decorin, lumican, biglycan, keratocan, syndican, agecan, perlecan, asporin, fibromodulin, epiphycan, PG-Lb, dermatan sulfate proteoglycan-3, versican, mimecan and mixtures thereof. Alternatively, the retained hydrophilic moiety can be a polymer selected from the group consisting of polyvinyl alcohol polyacrylic acid and mixtures thereof In other embodiments, the retained hydrophilic moiety is a biocompatible polymer with ionizable groups having a fixed charge density of about 0.01 to about 0.2 mEq/m³.

As described above, in certain embodiments, the method includes the step of contacting the collagen construct with a solution comprising a glycosaminoglycan or proteoglycan. Where the solution comprises a glycosaminoglycan, the glycosaminoglycan can be selected from the group consisting of hyaluronan, chondroitin sulfate, dermatan sulfate, keratan sulfate, heparin, heparin sulfate, and mixtures thereof. Where the solution comprises a proteoglycan, the proteoglycan can be selected from the group consisting of decorin, lumican, biglycan, keratocan, syndican, aggrecan, perlecan, asporin, fibromodulin, epiphycan, PG-Lb, dermatan sulfate proteoglycan-3, versican, mimecan and mixtures thereof In other embodiments, the method includes the step of contacting the collagen construct with a solution comprising a protein selected from the group consisting of collagen type IV, laminin, fibronectin, vinculin, an integrin moiety, and mixtures thereof.

The foregoing and other features and advantages of the system and method for producing a multilayer construct of aligned polymer structures will be apparent from the following more particular description of preferred embodiments of the system and method as illustrated in the accompanying drawings in which like reference characters refer to the same parts throughout the different views. The drawings are not necessarily to scale, emphasis instead being placed upon illustrating the principles of the invention.

### BRIEF DESCRIPTION OF THE DRAWINGS

FIG. 1 illustrates a preferred embodiment of the apparatus used to generate a single layer of oriented self-assembled polymer molecules in accordance with the present invention.

FIG. 2 illustrates a flow chart for producing aligned collagen fibers in accordance with a preferred embodiment of the present invention.

FIGS. 3A and 3B illustrate electron micrographs of collagen fibers prepared by a spin-coating method where FIG. 3A particularly illustrates random alignment of fibers and FIG. 3B illustrates fibers aligned in flow field in-accordance with preferred embodiments of the present invention.

FIGS. 4A and 4B illustrate a preferred embodiment of the apparatus used to generate a matrix of orthogonally-aligned layers in accordance with the present invention.

FIGS. 5A-5C illustrate another preferred embodiment of an apparatus used to generate a matrix of orthogonally-aligned layers in accordance with the present invention.

FIGS. 6A-6C illustrate schematically a folding method to generate a multilayer structure with orthogonally-aligned layers in accordance with a preferred embodiment of the present invention.

FIG. 7 schematically illustrates collagen fibers in the lamellae of the stroma in accordance with a preferred embodiment of the present invention.

FIG. 8A illustrates an apparatus having an offset holder wherein a monomer solution is deposited onto the substrate at the center of the offset disk holder in accordance with a preferred embodiment of the present invention.

FIG. 8B illustrates a flow chart of a preferred embodiment for producing aligned collagen fibers in accordance with a preferred embodiment of the present invention.

FIG. 9 is a scanning electron microscope (SEM) image demonstrating the deposition of a plurality of thin aligned layers onto a glass substrate in accordance with a preferred embodiment of the present invention.

FIG. 10 is a scanning electron microscope (SEM) image of a single layer of predominantly aligned collagen fibrils in accordance with a preferred embodiment of the present invention.

FIG. 11 is a scanning electron microscope (SEM) image of aligned collagen fibrils generated by the spin-coating methodology in accordance with a preferred embodiment of the present invention.

FIG. 12 illustrates a scanning electron microscope (SEM) image demonstrating layering of collagen in pseudolamellae in accordance with a preferred embodiment of the present invention.

FIG. 13 is a scanning electron microscope (SEM) image illustrating the interaction of two individual layers of aligned collagen in accordance with a preferred embodiment of the present invention.

FIGS. 14A-14C schematically illustrates a nanofabrication system and a flow-focussing method to manufacture layered, aligned polymer structures in accordance with a preferred embodiment of the present invention.

FIG. 15 schematically illustrates another preferred system to manufacture layered, aligned polymer structures in accordance with a preferred embodiment of the present invention.

FIGS. 16A and 16B illustrate a block diagram and a schematic diagram, respectively, of a preferred embodiment system to manufacture layered, aligned polymer structures in accordance with a preferred embodiment of the present invention.

FIG. 17A is a graphical representation of the relationships between positions within the layer or layer thickness on the ordinate and radial position and/or velocity on the abscissa for conditions of 750 rpm (dashed lines) and 1600 rpm (continuous lines) with constant addition of collagen solution (0.1 ml/min) to the center of a 2.5 cm radius disk in accordance with a preferred embodiment of present invention.

FIG. 17B is a graphical representation 520 of the relationships between shear rate or velocity of fluid or the ordinate and normalized position in layer at 25 mm from center on the abscissa for conditions of 750 rpm (dashed lines) and 1600 rpm (continuous lines) in accordance with a preferred embodiment of present invention.

FIG. 18 is a scanning electron microscope (SEM) image illustrating the interaction of two individual layers of aligned collagen in accordance with a preferred embodiment of the present invention. The image shows fibril alignment 2.0 cm from the fluid deposition point; with a flow rate of 0.25 ml/min and the rotation velocity of 1600 rpm. The top layer in this particular area formed incompletely due to de-wetting during the film formation, and the lower, orthogonal layer of fibrils can be seen passing under the top layer.

FIG. 19 schematically illustrates an Ussing-style perfusion chamber and a nanostructured artificial template (NAT) comprising collagen placed in between the half-chambers with a pressure applied to apply strain to the structure in accordance with a preferred embodiment of present invention.

FIGS. 20A-20C illustrate the flow chart of a method for producing a templated extracellular matrix comprising at least one population of cells from a nanostructured artificial template in accordance with a preferred embodiment of the present invention.

FIG. 21A schematically illustrates a flow chamber for generating layered aligned polymer in accordance with a preferred embodiment of present invention.

FIG. 21B is a top view of the flow chamber illustrated in FIG. 21A.

FIG. 22 illustrates the flow chart of a method for making a collagen-based implant material during the application of stress in accordance with a preferred embodiment of the present invention.

FIG. 23A schematically illustrates the corneal strip preparation. FIG. 23B schematically illustrates the results of enzymatically selecting collagen fibrils oriented parallel to an applied tensile load as demonstrated by birefringence in accordance with a preferred embodiment of the present invention.

### DETAILED DESCRIPTION OF THE INVENTION

The systems and methods of the present invention relate to processes and resulting material, suitable for the guided production of the self-assembly of aligned polymers in layers, and structures having multiple, non-aligned layers. More particularly the systems and methods of the present invention relate to the production of self-assembled biocompatible non-aligned thin lamellae.

This invention will be better understood with reference to the following definitions:

“Self-assembly”: a process by which a system spontaneously combines a number of smaller structural elements to form a large molecular or supramolecular complex. Self-assembly is the process by which many biological complexes form, such as DNA and collagen. This reaction sometimes requires that a threshold temperature, solution condition, or pH is reached before the spontaneous reaction occurs.

“Gel”: a material that is crosslinked through bonds of sufficient strength that it cannot be dissolved in ambient conditions. The bonds can be covalent, ionic, physical, or other.

“Stroma”: the supporting tissues or matrix of an organ, such as a connective tissue framework.

“Collagen”: a self-assembling biopolymer produced by reacting monomeric collagen in a neutral pH, slightly ionic solution. Collagen is a structural building block in the body, and is found in the cornea, skin, bones, blood vessels, ligaments, tendons, and cartilage.

“Polymerization”: the process by which monomer-sized molecules are assembled to form supramolecular structure. This process can occur through the formation of permanent chemical bonds, ionic bonds, or associative bonds. Fibrillogenesis is a form of polymerization in collagen.

“Polymer”: any supramolecular structure comprised of repeating subunits. These structures can be naturally occurring, such as proteins, or man-made, such as polyolefins.

A preferred embodiment of the invention comprises a method to produce a highly oriented, thin film of self-assembled polymer. The method comprises the process of subjecting a solution of monomeric building blocks of the self-assembling polymer to a shearing flow. While under the influence of this shearing flow, the polymer self-assembles, forming oriented polymeric structure in a thin film from approximately 500 nm to 100 μm thick. The preferred embodiments of the present invention provide for several processes of generating this shearing flow, examples of which are described herein below. The systems and methods of the present invention also provide for a process of forming constructs with multiple layers of aligned polymer structures, each layer having a different principal direction of orientation. The preferred embodiments provide for a method of forming constructs in both biopolymers and synthetic polymers.

### EXAMPLE 1

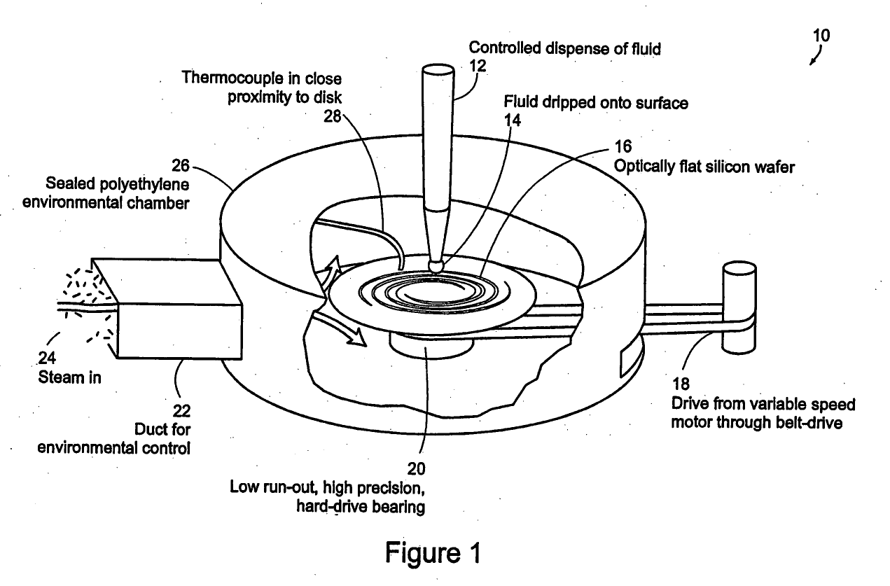

As one example of a preferred embodiment of this present invention, a metal disk is used as a deposition substrate. The disk is mounted on a spin-coating-type apparatus. A chamber is built around the apparatus to control the temperature and the relative humidity. The disk can be spun at a specified rate. This embodiment is illustrated in FIG. 1.

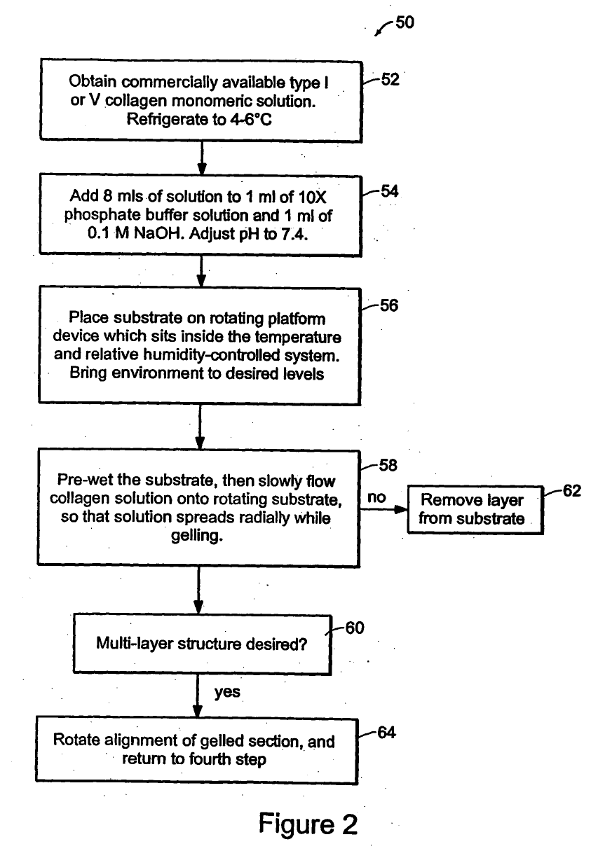

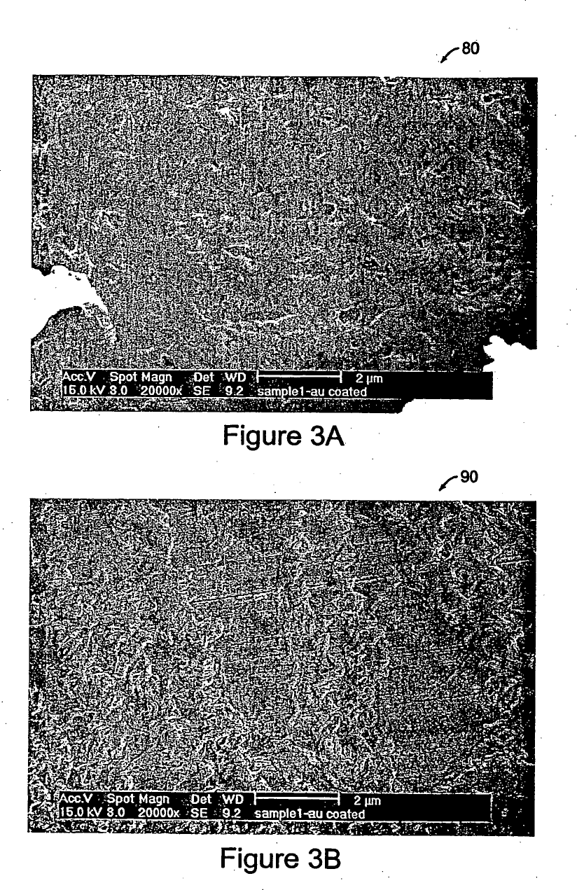

A solution of type I (Vitrogen™) collagen is chilled to 4-6° C. Bight ml of the collagen solution is mixed with 1 ml of 10× phosphate-buffered saline solution (0.2 M Na₂HPO₄, 1.3M NaCl, pH=7.4) and 1 ml of 0.1M NaOH. The pH is adjusted to 7.4±0.2 by adding 0.1M HCl. The solution is warmed to the test temperature, then steadily dripped onto the rotating substrate 16. The collagen gels form a uniform sub-micron thick sheet. This process is described with respect to the flow chart illustrated in FIG. 2. Nematic stacks are prepared by cutting out sections of the radially-aligned collagen fiber sheets, and stacking them orthogonally by hand. Electron micrographs of randomly oriented collagen fibers and oriented collagen fibers in a single layer prepared by this method are shown in FIGS. 3A and 3B, respectively.

### EXAMPLE 2

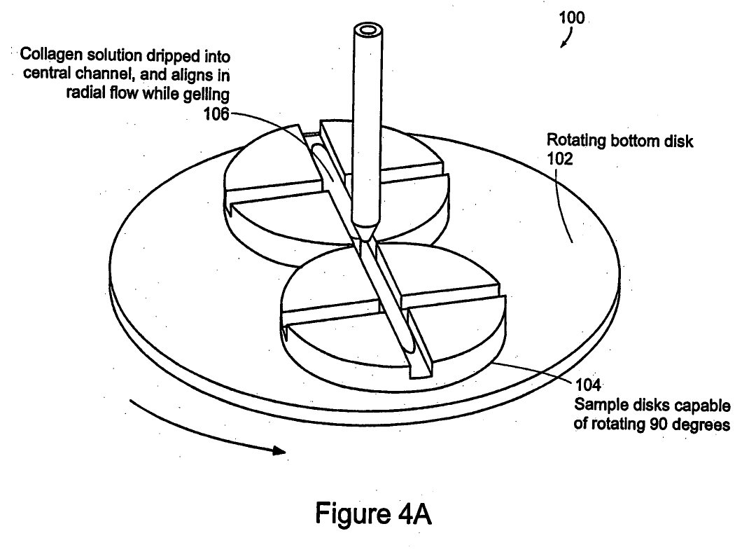

As a second example of a preferred embodiment of the present invention, sample disks 104 with T-slots cut into their surfaces are mounted on a disk 102 rotating at a specified rate as shown in FIGS. 4A and 4B. In accordance with a preferred embodiment of the present invention, the first layer of aligned associating fibrils is prepared by flowing the solution into channel of sample disks, which are on the rotating bottom disk. The centrifugal motion generates a shearing flow, which produces a thin layer and aids in aligning the growing associating polymers. The sample disks are then rotated 90 degrees, or to another specified angle, and the second layer is applied, which aligns with the specified angle relative to the first. A solution of Vitrogen™ collagen is chilled to 4-6° C. Eight ml of the collagen solution is mixed with 1 ml of 10× phosphate-buffered saline solution (0.2 M Na₂HPO₄, 1.3M NaCl pH=7.4) and 1 ml of 0.1M NaOH. The pH is adjusted to 7.4±0.2 by adding 0.1M HCl. The solution is warmed to the test temperature, then steadily dripped onto the center of the rotating disk, so that the solution travels down the channels extending to the outer radius of the rotating disk. After the first layer has gelled into an oriented collagen film, the sample disks are rotated 90 degrees, and the second layer is applied, so that the orientation direction of the second layer is 90 degrees with respect to the first layer. This process is repeated many times to generate a matrix of orthogonally-oriented collagen fibril layers.

### EXAMPLE 3

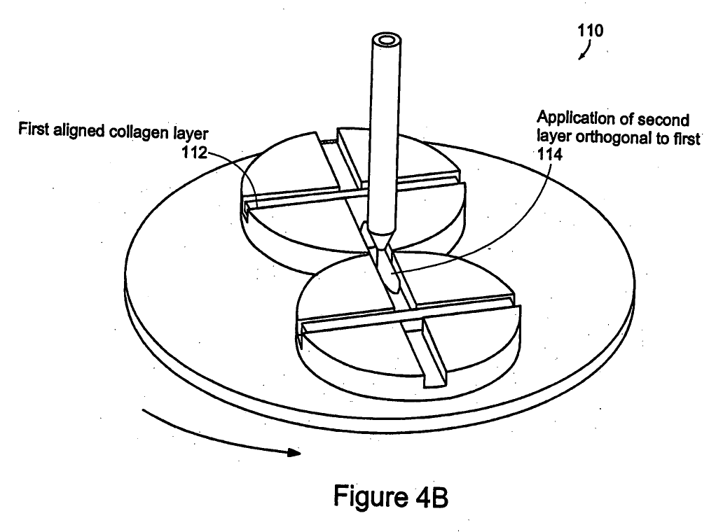

Another preferred embodiment of the present invention includes the monomer solution being placed in a temperature-controlled bowl as shown in FIGS. 5A-5C. In FIG. 5A, the polymer solution is subjected to a shearing flow by rotating the ball in one direction, or by oscillating in the same rotation plane. The polymer associates during this process. In FIG. 5B, the ball is rotated or oscillated in the orthogonal plane, to generate a layer of associated polymers orthogonal to the first layer. In FIG. 5C a three-dimensional (3D) rendering of the method is illustrated. A ball 122 with a diameter a few microns smaller than the bowl 128 diameter is placed in the bowl. A shaft 126 attached to the ball rotates the ball first in one direction, generating a shearing flow, during which time the monomer polymerizes. After a designated gelation period, fresh monomer solution may be introduced to the bowl, and the ball is rotated in the orthogonal direction, creating a layer orthogonally-aligned to the first layer.

### EXAMPLE 4

As another embodiment of the material described in this invention, highly oriented collagen layers prepared as described herein can be bundled to form the annulus fibrosus found in the spinal intervertebral disk. This disk acts primarily as a weight-bearing and flexible joint. The load bearing capability and flexibility in selected directions is achieved by the combination of the annulus fibrosus and nucleus pulposus. Annulus fibrosus is a layered structure that is rigid in the radial direction but deformable in the axial direction and by torque. This structure has alternating layers of oriented collagen fibrils, similar to that described in the cornea. Each layer has its collagen fibrils wound at an angle, and subsequent layers have an alternate orientation. Such a structure helps to achieved a maximum resistance to radial stress, while allowing a deformation in torque and bending.

### EXAMPLE 5

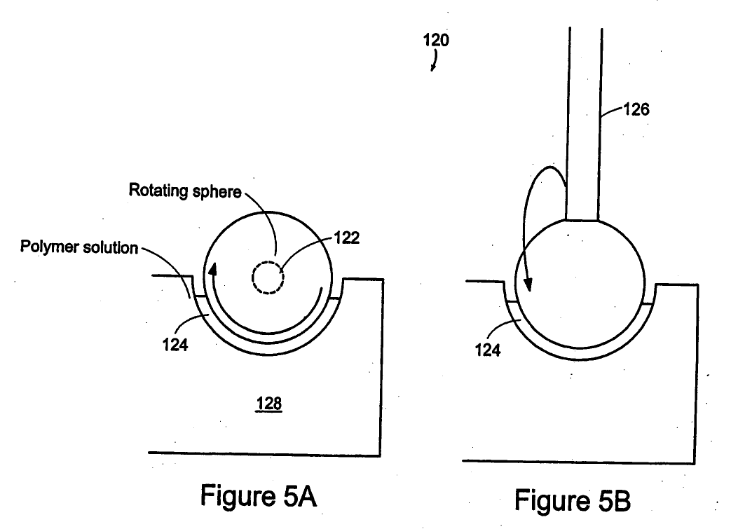

As another preferred embodiment of this invention, single layers of aligned polymer structure are generated with the techniques and methods discussed herein. The layers are folded as shown in FIGS. 6A-6C, to generate successively crossed layers of aligned polymer fibrils or other superstructure.

The extracellular matrix of the mature intact cornea comprises an extremely varied yet highly structured array of collagens, proteoglycans, glycoproteins and soluble macromolecules.

Current attempts to generate a biomimetic corneal construct have yielded corneas that behave similarly to in vivo corneas with respect to the function of the cellular layers. However, these corneal constructs have severe limitations with regard to the structure of the stromal extracellular matrix (ECM), which was constructed to ensure biocompatibility, nominal transmittance and the ability to promote adherence of the superficial cell layers and little more. It appears that previous investigators focused on the cellular layers but lost sight of the importance of the stroma itself. The three major functions of the cornea: protection, refraction and transmission, are performed primarily by the stromal ECM, the structure of which is optimally designed to accomplish these objectives. Thus, the cellular layers serve only to maintain and defend the stroma, which provides the principal functions of the cornea.

Strength. The major structural collagen of the stroma (type I collagen) is arranged in 300 to 500 lamellae of parallel, non-crosslinked fibrils. The lamellae are stacked in the anterior posterior (AP) direction and the fibrils of adjacent lamellae are nearly perpendicular to each other. Free ends of the fibrils have not been discerned in the cornea, which suggests that they run uninterrupted from limbus-to-limbus. This “plywood” arrangement of the lamellae gives the cornea remarkable strength tangential to the surface. Randomly crosslinked collagen networks of similar thickness (used in current constructs) cannot provide similar yield strength.

Refraction. The ability of the fibrils to slide relative to each other with ease allows the natural cornea to distribute the load imposed by the intraocular pressure (IOP) uniformly. This enables the anterior surface of the eye to form a nearly perfectly spherical shape for refraction. Stromas comprising randomly cross-linked collagen are likely to form imperfect surfaces for refraction upon inflation to normal IOP. When these random networks are loaded, complex stress fields are formed that result in inhomogeneous refraction and thus compromised optical qualities.

Transmission of light. The uniform diameter of natural corneal fibrils and their short-range ordering allows light to pass through virtually unimpeded. Randomly crosslinked collagen networks, though nominally transparent, cannot produce the same quality optical properties. The process described in the embodiments of this invention can produce orthogonally stacked arrays of aligned type I/type V collagen fibrils. The general biomechanical and optical properties of such a construct can be similar to native corneal stroma.

The difficulties in forming a fully complete, functional, corneal stroma, constructed de novo, by artificial means have led some to indicate that it may not be a viable given the technology available today. However, it is not likely to be necessary to complete the construction of the stroma purely by mechano/chemical manipulation of biochemical components in the laboratory. A partially completed primary corneal stroma comprising orthogonal layers of aligned type I/type V heterotypic collagen fibers can suffice as a suitable scaffolding or starting point, as it does during embryo genesis.

In support of this approach, it is known that following full-thickness trephination wounds in rabbit corneas, the healing response is capable of transforming the initially opaque fibrous plug to tissue that is similar to normal corneal stroma. The initially opaque scars comprise large interfibrillar spaces, unusually large chondroitin sulfate (CS) proteoglycans, hyaluronic acid and no detectable keratan sulfate proteoglycan. After one year of healing and remodeling, normal interfibrillar spacing, size and distribution of proteoglycans is restored. This mechanism works even if tissue of similar biochemistry, but not structure is implanted into the cornea. It has been found that the corneal healing response has the capacity to partially resolve collagen fibril structure even when scleral tissue is used to repair corneal wounds (Winkelman 1951 Am J Ophthalmol; Kurz 1953 Cs Oftal; Maurice and Singh Cornea 1996) incorporated herein by reference in its entirety.

The present invention takes advantage of the natural healing response and remodeling ability of the corneal stroma. A stromal scaffolding remodeled by the corneal wound healing response, can be implanted. However, implanting a biomimetic stroma that is not sufficiently strong, transparent, or smooth is not acceptable. The artificial stroma, at the outset, must preserve the optical qualities of the cornea during the remodeling period. Current corneal constructs, which employ randomly cross-linked type I collagen fibrils, cannot meet this requirement The closer the tissue or scaffolding to be remodeled mimics native stroma, the less time it takes to fully resolve the structure and fully incorporate the graft. Further, if the initial scaffolding is capable of performing the three major functions of the stroma (protection, refraction and transmission), remodeling in vivo may proceed while vision remains clinically acceptable.

Thus, from investigations into the developmental biology of the chicken, it has been learned that corneal embryo genesis is an intricately orchestrated and complex phenomenon. Initially, from superficial epithelial cells derived from an offshoot of the developing brain, an orthogonally organized primary corneal stroma of type I collagen is secreted layer-by-layer. The extracellular matrix (CM) of the mature intact cornea comprises an extremely varied yet highly structured array of collagens, proteoglycans, glycoproteins and soluble macromolecules.

With regard to biomimetic corneal constructs, recent attempts have been made to develop full corneal constructs de novo. These efforts have yielded corneas that behave similarly to in vivo corneas with respect to the function of the cellular layers. However, these corneal constructs have severe limitations with regard to the structure of the stromal ECM, which was constructed to ensure biocompatibility, adequate transmittance and the ability to promote adherence of the superficial cell layers and little more.

Completely artificial corneal replacements, such as the K-pro, have not met with widespread clinical success. Such devices are not yet qualified for use in clinical situations where a transplant would be considered even marginally effective because of the high potential for devastating complications and the need for continual high quality follow-up. Current incarnations of corneal constructs generated from biomimetic materials and live cell layers do not include physiologically, ultrastructurally or biochemically realistic stromas. In addition there have been a number of efforts towards the production of an artificial cornea which have not met with complete success.

With regard to effective scaffolding and wound healing response, a fully complete, functional corneal stroma, constructed de novo, by artificial means may not be a viable approach given the technology available today. However, it is not likely to be necessary to complete the construction of the stroma purely by mechanical or chemical manipulation of biochemical components in the laboratory. A partially completed primary corneal stroma comprising orthogonal layers of aligned type I/type V heterotypic collagen fibers might suffice as a suitable scaffolding or starting point, as it does during embryo genesis. Such a solution would be a marked improvement over current systems, allowing easy cell infiltration and critically, immediate functionality both optically and mechanically.

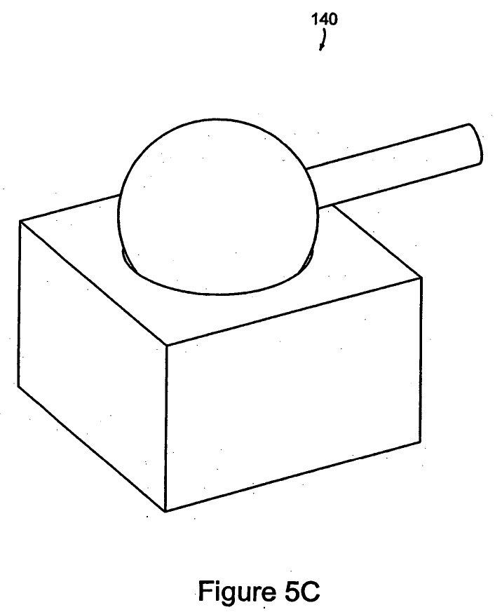

FIG. 7 illustrates collagen fibers in the lamellae of the stroma in accordance with a preferred embodiment of the present invention. The behavior of associate rods (collagen) in solution is explored. Collagen fibrils are composed of triple-helix collagen macromolecules that are approximately 300 nm long and 1.5 nm in diameter. These segments are produced inside cells and are excreted as procollagen whereupon the ends are removed to form a macromolecule that naturally self-assembles. Although there is some debate over the exact mechanism of this self-assembly process, it is understood that the assembly of collagen macromolecules into fibrils is an entropy driven process. The most favorable structure is therefore a cylinder 184 which minimizes the surface area. This fibril assembly is very similar to other protein polymerizations. Each of these cylindrical bundles is composed of segments of associated molecules in the familiar 67 nm repeating structure. There is still some debate over the exact molecular process of association but it appears that the cylindrical bundles of segments end-associate whereby the terminating C-telopeptide 192 associates with the N-telopeptide on the adjacent chain The telopeptides also influence the diameter of the fibril and the segment packing, but there are also probably influences from glycosaminoglycans (GAGs), proteoglycans, solution temperature and concentration. All of these components can be controllable in a manufacturing process. Further, there is some evidence that the association is driven by a further hydrophobic effect between the telopeptides. These fibrils then associate to form fibers.

Thus with no other influence, a solution composed of collagen macromolecules (i.e. the basic triple helix unit) forms an isotropic random gel. However, with the correct driving force, these gels can be forced to grow in an aligned manner. Magnetism, drainage flows and confining effects have been used successfully, but only weakly influence the morphology of the resulting gels.

Orientation of a rigid-rod in a flow field is determined by the balance of hydrodynamic forces (aligning) and rotary Brownian motion (randomizing) which is described by the Peclet number Pe=II_{2D}/Dᵣ, where Dᵣ is the Brownian diffusion coefficient and II_{2D} is the convective diffusion coefficient. Simple estimates suggest that the flow field can orient the collagen monomers because of their size. (Pe<1) but that in fact for the monomers the flow is very close to the transition ranges. However, as the monomers associate and the collagen fibrils grow, the aspect ratio increases and consequently the Peclet number increases rapidly as it is related to the cube of the length of the rod.

$$Pe = \frac{{II}_{2 D}^{1 / 2}}{{D}_{r}} = \frac{\dot{γ}}{{D}_{r}} \qquad Equation 1$$

For thin prolate spheroids (rₚ>>1) in shear flow

$$Pe = \frac{{II}_{2 D}^{1 / 2}}{{D}_{r}} = \frac{\dot{γ}}{{D}_{r}} = \frac{8 π {η}_{s} {a}^{3} \dot{γ}}{3 kT ( 1 n ( 2 {r}_{p} ) - \frac{1}{2} )} \qquad Equation 2$$

wherein

$${r}_{p} = \frac{a}{b}$$

is the aspect ratio of spheroid (a is long dimension). At 300 K for rigid rods of length 300 nm and diameter 30 nm in a solvent of 1 mPa.s and at a shear rate of 600 s⁻¹ the Peclet number is 13.

In addition, the proximity of walls also act to orient the monomers in the plane of the wall. This influence is especially strong in confined films that are only an order of magnitude larger than the longest axis of the collagen monomers. Concentrated solutions of rigid non-interacting rods are known to behave in a liquid crystalline fashion, forming nematic structures where there is long range order in the direction of the long axis of the rod. In theory, the direction of this ordering is arbitrary but is usually generated by perturbations, such as the confining effect of a wall, or solvent flow. Although nematic structures built from non-interacting rods require a critical concentration to transition from an isotropic to a nematic phase. In the case of interacting rods, the situation is more complex, but the interactions can clearly influence the ability of the molecules to align. Once fibrils have begun to grow the flowing monomers passing across the substrate in the shear field naturally end attach since there is a pronounced anisotropy between side addition and end addition for interacting rods. In addition magnetic (or electric) fields are known to strongly align nematic liquid crystals and have been proven to influence the alignment of growing collagen gels.

With regard to the alignment of polymers, it is well known that flow aligns polymers. Both shear and extensional flows align polymers in different manners but in general if the flow is strong enough there is some form of alignment in the direction of the flow. Spin-coating, and other similar flows, such as film drainage and steady shear, have been observed to produce roughly oriented polymers from the melt, for rigid-rod molecules and for liquid crystals. Alignment in these systems is often however only local and is usually for systems where the macromolecules themselves are being aligned. The alignment of growing fibers or polymers is less well recognized a process.

It has also been observed that shear induces alignment in polymers during the polymerization process of rod-like molecules. However simple shear does not provide sufficient force to counter the random fluctuations of the chains, and their natural propensity move under Brownian motion. The preferred embodiments of the present invention use spin-coating methods to provide a flow regime suitable for initiation and growth of aligned collagen fibrils. The solution conditions can be modulated while the polymerization process is proceeding, and the naturally radial nature of the flow assists in maintaining the fiber separation and promotes growth from a central core area. In addition the spin-coating process provides a natural length scale that can be easily adjusted to control the layer thickness and rate of polymerization. Also, since it is known that the proximity of a surface influences the position of an adjacent molecule, the thin confinement layer promotes alignment in the most critical direction, parallel to the layers longest axis. Spin-coating has been used to produce thin homogeneous films for a number of years. In melts and liquid crystals this deposition process is known to align polymers. This method is extremely well understood for evaporating and non-evaporating solvents since it is used heavily commercially in silicon microfabrication processes and in the optics industry.

With regard to the alignment of collagen, examples of artificial alignment of collagen are relatively common in the literature, but rarely result in the highly aligned configuration that is required for a truly biomimetic system. Previously noted parameters such as gravity, shear, diffusion and other external forces can influence the morphology of the fibers, but in all cases the alignment is weak. Other observations include alignment of collagen-like self-assemblies using shear while cooling from a hot solution. All of these cases require relatively concentrated solutions which limit the control over the fiber morphology. In addition collagen has been aligned using the natural propensity of collagen monomers to self-assemble in concentrated solutions. However, this method allows no control of the fiber sizes and shapes. To provide the highly aligned morphology two things are critically required, a thin confinement layer, and a unidirectional flow. Spin-coating provides both requirements readily. Although spin-coating has been observed to generate alignment in thin films, these are usually from melts and in polymerized systems. It should be noted that alignment as traditionally detected for many of these systems, using circular dichroism, does not indicate alignment on the levels required by a corneal analog.

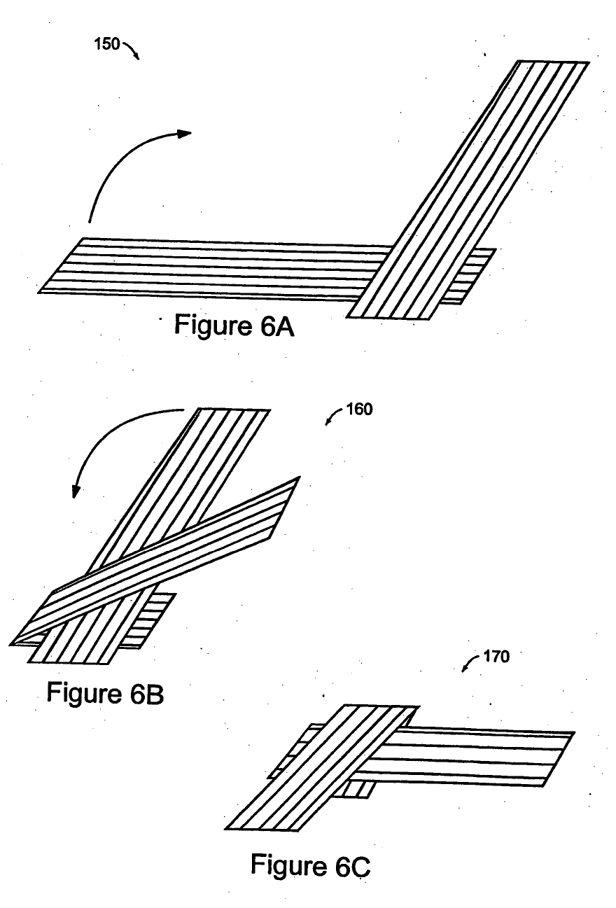

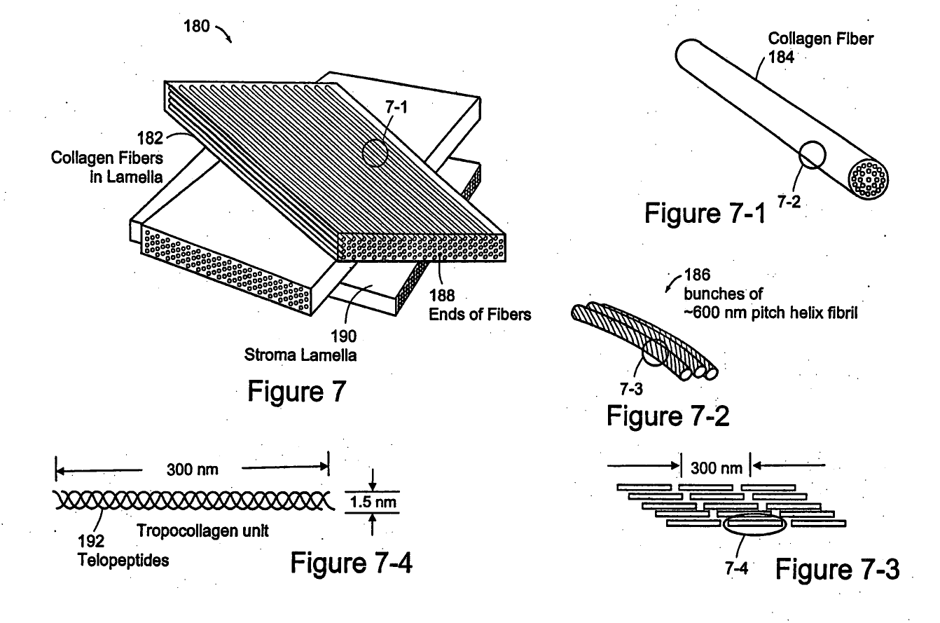

FIG. 8A illustrates an apparatus having an offset holder wherein a monomer solution is deposited onto the substrate at the center of the offset dish holder in accordance with a preferred embodiment of the present invention. FIG. 8B illustrates a preferred methodology to create single and multiple layers of aligned polymer films in accordance with a preferred embodiment of the present invention. In this system and method a substrate is placed off-center on a substrate holder. The holder 206 is rotated at a prescribed velocity or sequence of velocities to create a thin shearing flow. The monomer solution 202 is added to the deposition substrate 210 (either by steady flow or by unsteady dripping) and is carried radially away from the injection point by the centripetal acceleration. Thus a single layer (film) of aligned collagen fibrils is created. To generate multiple layers with different alignment directions, the deposition substrate 210 is rotated at any prescribed angle and another layer may be deposited.

FIG. 8B is a flow chart describing method 216 for generating aligned collagen via spin-coating in accordance with a preferred embodiment of the present invention. The method begins with step 218 of obtaining commercially available type I collagen monomers. They are kept refrigerated at 4-6° C. Extracted or recombinant human and/or animal collagen type I monomers may be used. Collagen type I monomer concentrations are in the range of 0.01 mg/ml to 100 mg/ml with a preferred range of 0.5 to 10 mg/ml. A preferred embodiment utilizes Vitrogen (brand) bovine collagen (3.0 mg/ml). In an alternate embodiment, if heterotypic fibrils are being made for diameter control, type V collagen monomer is obtained as well. Extracted or recombinant human and or animal collagen type V monomers may be used.

Per step 220, collagen type I is prepared for polymerization. In a preferred embodiment, Vitrogen is neutralized in preparation for self-assembly by adding 8:1:1 ratio of collagen type I:10×PBS: 0.1M NaOH. The pH is adjusted to 7.4. If type V is also included, the solution is further processed to ensure neutralization of type V as well. In preferred embodiments, additives may be used to alter the viscosity of the collagen solution to change the shear stress on the growing fibrils. Such additives include, for example, but are not limited to, glycerol. The viscosity of the final solution of monomer is in the range of 1.0 mPa.s to 100.0 Pa.s. The preferred range is from 5.0 mPa.s to 1.0 Pa.s. In a preferred embodiment, the viscosity of the collagen monomer solution is approximately 10 mPa.s.

The method 216 in accordance with the preferred embodiment includes the step 222 of substrate selection The substrate that accepts the collagen coating may comprise of any material that can be generated with a uniform optically flat surface to promote the establishment of a uniform shear flow field during spin-coating. Asperities on the surface of the material may be in a range of 0 to 10 micrometers, with a preferred range of 0.1 to 0.5 micrometers. A preferred embodiment, for example, utilizes a 2 inch diameter borosilicate glass disk.

The method 216 also includes the step 224 of preparation of the substrate to be coated with collagen. Preparation may include modulation of the surface of substrate to be uniformly hydrophilic, uniformly hydrophobic, gradient hydrophilic, or to preferentially bind collagen, for example, antibody inclusion. A preferred embodiment for hydrophilic treatment for glass substrate utilizes, for example, 1.5 hour ultrasonication of substrate in 10% Micro90™ cleaner. Glass is stored in deionized water until use.

Per step 222, the substrate to be coated with collagen is positioned into the device designed to generate centripetal acceleration. In a preferred embodiment, the substrate is placed directly onto a vacuum chuck of a commercial spin-coater.

If multiple layers of collagen oriented at differing relative angles are desired another substrate handling system is required, for example, in a preferred embodiment a borosilicate glass disk is placed in an offset disk holder of FIG. 8 or a system as shown in FIG. 4 is utilized.

The method 216 includes the step 228 of modulating the environment surrounding the substrate to create conditions conducive to initiate polymerization of collagen. The environmental conditions include an ambient air temperature range of about 25° C. to about 45° C., preferably about 35° C. to about 42° C. and about 80-100% ambient air humidity with a preferred range of 90-100% humidity. In a preferred embodiment, a steam heat humidifier is attached to vent ports in the housing of the substrate rotating device as shown in FIG. 1. In alternate embodiments, the substrate or substrate holder may also incorporate a heating device to locally control temperature of the substrate surface. In one embodiment, the substrate is heated by an electric heater, such devices require rotating electrical contacts, for example, a commutator.

A preferred embodiment of the present invention includes pre-processing or pre-wetting the substrate. Prior to sustained addition of the monomer to the substrate, a bolus of cold monomer is added to the substrate to ensure that the substrate is fully wet In a preferred embodiment, an amount capable of covering the entire substrate is injected and is used to be spread on the substrate.

The method 216 further includes step 230 of initiating a flow of the monomer units. Prior to substrate rotation, a flow of monomer is begun. For neutralized collagen monomer solution, the start-up range is about 0.05 to about 1000 ml/min, preferably about 0.1 to about −100 ml/min. In a preferred embodiment, the start up flow rate for neutralized collagen monomer solution is about 2 ml/min.

The method then includes the step 232 of initiating rotation of the substrate. The rotation of substrate is initiated and may proceed in a series of steps to aid in the uniform spreading of the collagen monomer solution over the substrate surface. A range of initial angular velocities during startup is about 10 to about 5000 rpm with a preferred range of about 60 to about 2000 rpm. In a preferred embodiment the initial angular velocity utilized is about 250 rpm.

The method further includes the step 234 of controlling a plurality of parameters during polymerization. For example, the rotational velocity of the substrate during polymerization is controlled. During the polymerization of the collagen monomer on the warm substrate, the rotational velocity may remain steady or undergo modulation of any kind. However, the average velocity (based on averaging over each minute of operation) of the substrate has a range of 100 to 50,000 rpm with a preferred range of about 500 to about 10,000 rpm. In other embodiments, the average velocity is about 250 rpm to about 1600 rpm. In a preferred embodiment, the average rotational velocity of the substrate is about 1600 rpm.

Further, during polymerization, the flow rate of monomer is controlled. The monomer solution flow rate provides a monomer for polymerization to the substrate surface while generating suitable shear force under rotation to induce alignment of the growing collagen fibrils. The flow rate of collagen solution over the rotating substrate may remain steady or undergo modulation of any kind. However, the average flow rate of collagen solution (based on averaging over each minute during operation) is in a range of between about 0.05 ml/min to about 1000 ml/min with a preferred range of about 0.1 to about 100 ml/min. In a preferred embodiment, the average flow rate over the substrate during polymerization is about 0.25 to about 2.0 ml/min.

Another parameter that is controlled during polymerization is the optimum shear rate at the substrate interface. The flow rate over the growing monomers tends to align them along the flow direction and provides the monomer units access to the free end. The combination of monomer solution input flow rate, viscosity and substrate rotational velocity combines to produce a range of shear rates from 1 Hz to 500,000 Hz with a preferred range of about 50 to about 50,000 Hz. In a preferred embodiment, the shear rate at the substrate surface is about 700 Hz.

The duration of polymerization is another parameter that can be controlled. Rheological experiments have demonstrated that the gelation of the collagen solution begins once optimum conditions are achieved and take approximately six minutes following addition to a substrate warmed to 37° C. In the apparatus in accordance with a preferred embodiment of the present invention, the range of time of exposure of the monomer flow to the rotating substrate lies between 1 minute and 2 hours with a preferred range of 3 minutes to 1 hour. In a preferred embodiment, to generate a single layer of polymerized collagen, flow is sustained over the rotating substrate for about 15 to 20 minutes.

The method 216 in accordance with the present invention includes the step 236 of initiating a spin-down procedure. Following the addition of collagen to the rotating substrate, the rotating substrate continues spinning to remove excess collagen monomer. This spinning down procedure may include a rinse step where a solution containing no collagen monomer is applied to the rotating substrate to enhance the removal of unreacted monomer.

A layer of material can be added to separate collagen layers, for example, to promote cell attachment and proliferation. In preferred embodiments, a layer including collagen type IV and other proteins such as, for example, but not limited to, laminin, fibronectin, vinculin, an integrin receptor or mixture thereof is deposited between aligned polymer layers. During the spin-down procedure the rotational velocity may remain steady or undergo modulation of any kind. However, the average velocity (based on averaging over each minute of operation) of the substrate is in a range of about 100 to about 50,000 rpm with a preferred range of about 500 to about 10,000 rpm. In a preferred embodiment, the average rotational velocity of the substrate is about 1600 rpm.

The method 216 concludes with a post-processing step 238. To ensure polymerization of the deposited layer, the substrate may be post-processed. Post-processing may include an extended exposure to the warm humid environment for a period of 0.1 to 60 minutes with a preferred range of exposure of about 3 to about 10 minutes. In a preferred embodiment, the exposure time during post-processing is about 5 minutes. The method 216 includes all of the essential steps to produce a single layer of aligned collagen via spin-coating in accordance with a preferred embodiment of the present invention.

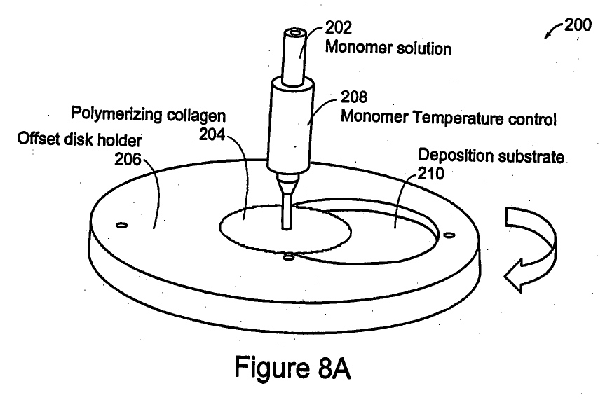

FIG. 9 is a scanning electron microscope (SEM) image demonstrating the deposition of a plurality of thin aligned layers 244 onto a glass substrate 242 in accordance with a preferred embodiment of the present invention.

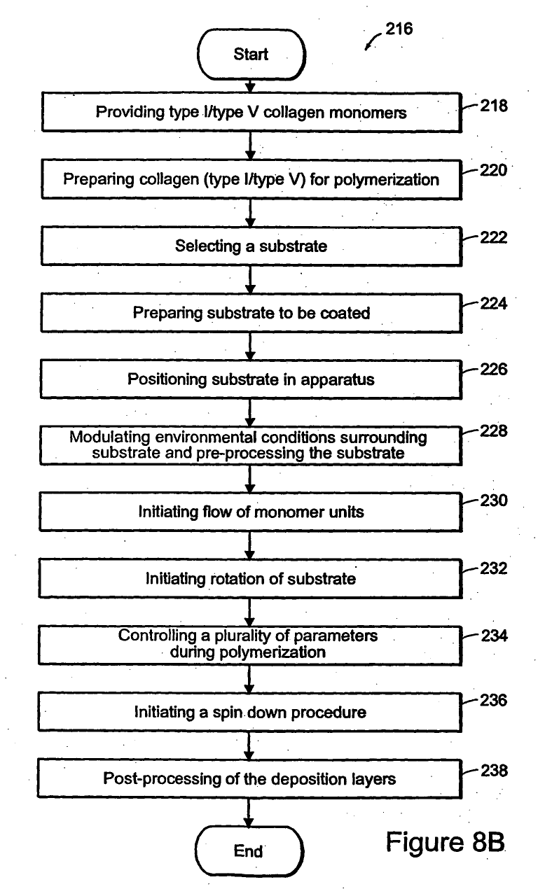

FIG. 10 is a scanning electron microscope (SEM) image of a single layer of predominantly aligned collagen fibrils in accordance with a preferred embodiment of the present invention.

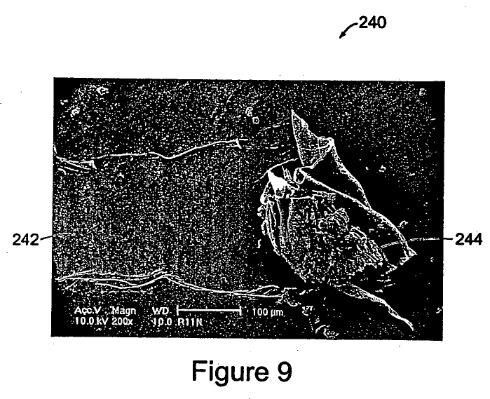

FIG. 11 is a scanning electron microscope (SEM) image of aligned collagen fibrils generated by the spin-coating methodology in accordance with a preferred embodiment of the present invention.

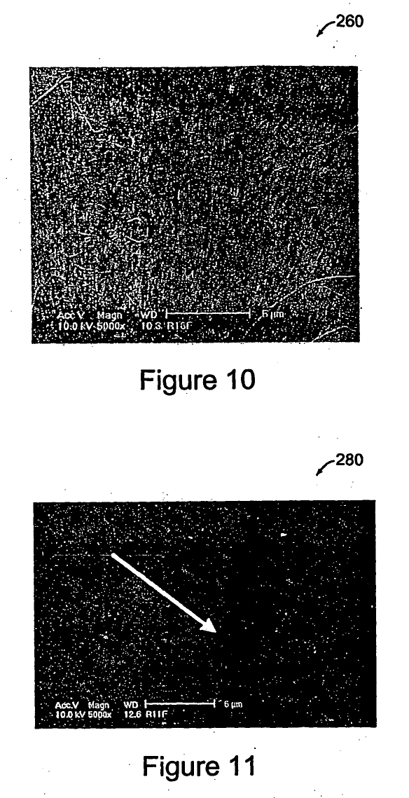

FIG. 12 illustrates a scanning electron microscope (SEM) image demonstrating layering of collagen in pseudolamellae in accordance with a preferred embodiment of the present invention. Multiple layers of aligned collagen polymer may be achieved by repeating the procedure for generating a single layer as many times as required. The method 216 may be repeated immediately following the post-processing step or repeated following a dryout period or a deionized water soak period of several minutes to several days. In a preferred embodiment, multiple layers are achieved by repeating the single layer procedure following a soaking period of about 24 hours in deionized water.

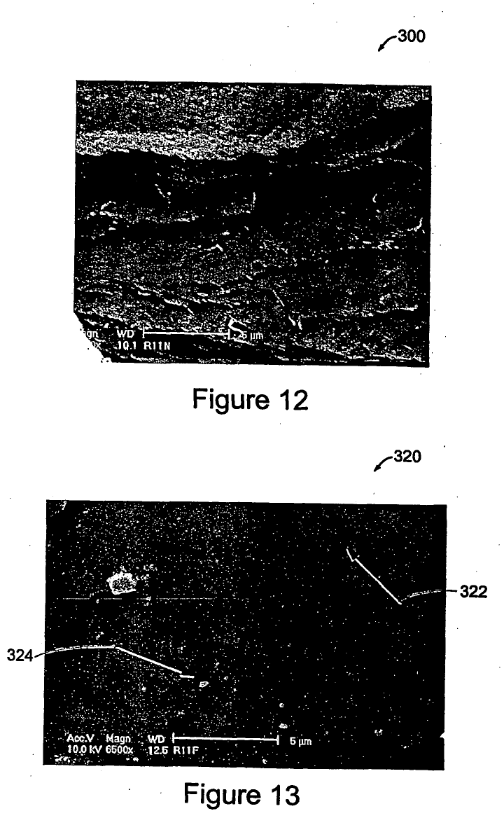

FIG. 13 is a SEM image 320 illustrating the intersection of two individual layers of aligned collagen in accordance with a preferred embodiment of the present invention. The arrows 322, 324 indicate the alignment directions for each layer. To generate multiple layers of aligned collagen polymer where the relative angle of alignment of the collagen is changed between depositions, the method 216 for producing multiple layers is performed. However, the substrate is rotated through any angle relative to its previous position on the substrate holder. Any range of angles is possible from a range of 0 to 2π (and any integer multiple thereof) with a preferred range of 0 to π. In a preferred embodiment, the angle between aligned collagen layers is π/2.

With regard to microfluidics methodologies, the fundamental principle in the embodiments of the present invention is to use flow regimes to control the growth of individual fibers of collagen, or some other polymerizable material. The method 216 outlined the use of a common industrial process, namely spin-coating, to provide the necessary constraints to the growing fibers to allow control of alignment, length and diameter. However in a more general embodiment it is possible to directly manipulate the flow field, and thus the local environment, around a growing filament. To this end the use of the emerging field of microfluidics can be used.

The use of microfabricated fluid handling is described by Giordano, N. and Cheng, J.-T. (2001) in the Journal of Physics: Condensed Matter, 13, R271-R295 entitled “Microfluid mechanics: progress and opportunities”, the entire teachings of which is hereby incorporated by reference. These methods have matured over the last few years resulting in commercial products such as DNA sorting systems. These systems can be used to handle fluids on sub-micron scales using features and channels that can be applied to sub-micron dimensions.

To control single growing filaments, the length scales required must be closer to the characteristic dimension of the filament, approximately 50 nm. Confinement in tubes rapidly allows extension of the filament and the narrow confines of such a channel rapidly increase the shear rates around the filament However these narrow channel sizes are currently difficult to manufacture and use.

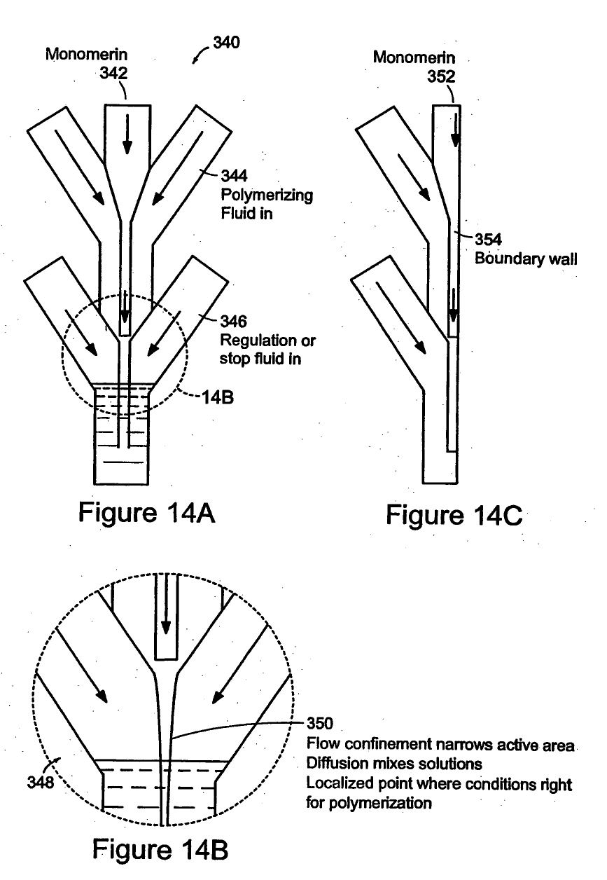

A solution is described by a preferred embodiment of the present invention and uses a form of flow focussing illustrated in FIGS. 14A-14C which provide a schematic of the flow focussing concept At the length scales discussed herein flow is almost always laminar. This means that in fact mixing is very difficult to encourage in such flow regimes. In reality then the mixing process is controlled by diffusion processes. Consequently, if input two jets impinge on each other at an angle greater than 1 degrees, they do not mix directly but must mix by diffusion across the interface. If one of these jets contains the predominantly monomeric solution (monomeric collagen or other species) discussed hereinbefore, and the other jet contains the polymerizing agent, then there is a finite time before enough diffusion occurs to allow mixing.

If the polymerizing jet has a higher volume flow rate and both jets enter the same size channel, then the amount of the channel used by each fluid “species” is proportional to the incoming flow rate. Consequently this behavior allows the fluids to control the active zone in the flow field, both through constriction of the width as shown in FIG. 14B, related to the relative flow rates (this is the flow focusing described hereinbefore), and through diffusion related to fluid concentrations.

The procedure outlined with respect to FIGS. 14A-14C allow a confined area to be generated in a flow field that can be arranged to have the correct solution conditions for polymerization. In addition other fluids can be added to influence the filament diameter and, if necessary, stop polymerization. If the growing polymer is advanced such that the growing tip is always in this critical region, the polymer can be extruded indefinitely. This approach allows the nanofabrication of a single polymer fiber.

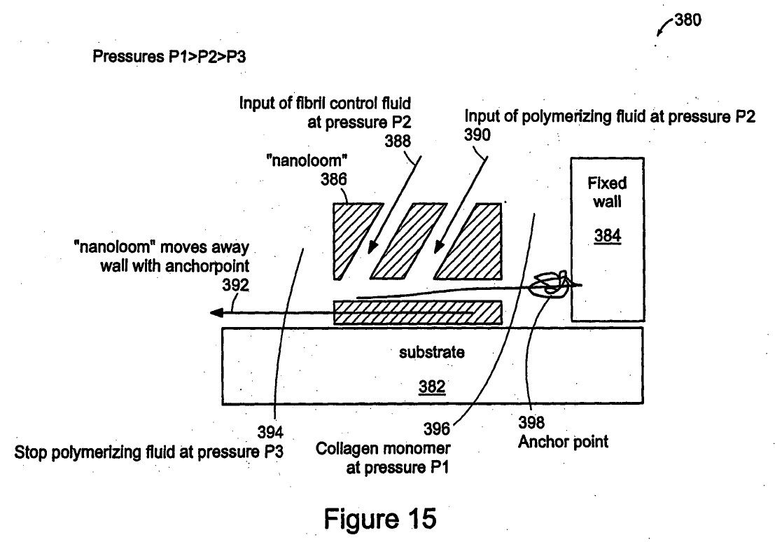

FIG. 15 illustrates another preferred system to manufacture layered aligned polymer structures in accordance with a preferred embodiment of the present invention. This preferred embodiment utilizes the recognition that if an array of channels such as described in FIGS. 14A-14C is manufactured an array of controlled polymers can be generated. In FIG. 15, initially the collagen is polymerized against a fixed wall 384 to ensure a dangling collagen chain is present in the nanoloom 386. As the collagen filament grows this nanoloom 386 advances relative to the fixed wall, thus extruding a single collagen filament as it advances.

An array of these filaments allow construction of a single layer of aligned collagen.

It may also be possible to produce woven materials in this manner.

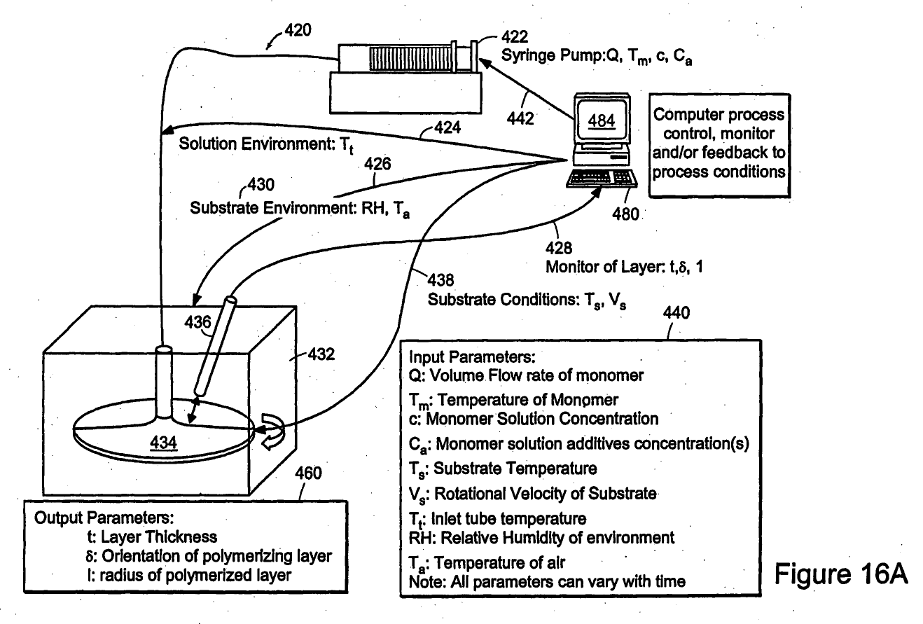

FIGS. 16A and 16B illustrate a block diagram 420 and a schematic diagram 490, respectively, of a preferred embodiment system to manufacture layered, aligned polymer structures in accordance with the present invention. The apparatus 432 used to generate layered, aligned polymer structures has a plurality of input parameters 440 that can be modulated using a processor 480 activating effectors, typically via D/A interfaces. The input parameters, include, without limitation, volume flow rate of monomer, temperature of monomer, monomer solution concentration, concentrations of monomer solution additives, substrate temperature, rotational velocity of substrate, inlet tube temperature, relative humidity of the ambient air of the environment, and temperature of the ambient air. It should be noted that all parameters can vary with time. A plurality of output parameters 460 are also monitored and modulated by the processor 480. The output parameters include, without limitation, layer thickness, orientation of polymerizing layer, and radius of polymerized layer. The system includes a plurality of sensing elements, monitoring elements and effectors that enable the processor 480 to process, control and monitor different parameters. The processor through an input/output interface 442 interfaces with a pump to monitor and/or control the volume flow rate of the monomer, the temperature of the monomer, the concentration of the monomer solution and the concentration of any additives to the solution. Further, the solution environment, for example, the temperature of the solution is monitored using the interface 424. The substrate environment, for example, the relative humidity and the temperature can be monitored and modulated using an interface 426. The substrate conditions are monitored such as, for example, substrate temperature and the rotational velocity of the substrate. The parameters associated with the layers of aligned polymers, for example, layer thickness, orientation of polymerizing and radius of polymerized layers are also monitored using an interface 428. The processor 480 can have an integrated display device or provide data to another display device and/or processor that is not co-located with the device 432. Post-processing of the polymer structure as described with respect to the method illustrated in FIG. 8B can be controlled by the processor 480. It should be noted that the device 432 can comprise a distribution network for the polymer solution that includes a plurality of nozzles that can be rotated and deposit the polymer/monomer solution onto a substrate that can be stationary. The shear flow is generated by a relative motion and/or velocity between the distribution system and the substrate.

The system 420 may also include a microprocessor and a memory device that stores display data. The microprocessor may include an operating system, as well as application and communication software to implement the functions with respect to controlling device 432 operation. The operating system for the system of the present invention includes a processing system with at least one high speed processing unit and a memory system. In accordance with the practice of persons skilled in the art of computer programming, the present invention has been described herein with reference to acts and symbolic representations of operations or instructions that are performed by the processing system. Such acts, operations and instructions are also referred to sometimes as being computer executed or processing unit executed.

It will be appreciated that the acts and symbolically represented operations or instructions include the manipulation of electrical signals by the processing unit. An electrical system with data bits causes a resulting transformation or reduction of the electrical signal representation, and the maintenance of data bits at a memory location in the memory system to thereby reconfigure or otherwise alter the processing unit's operation, as well as other processing of signals. The memory locations where data bits are maintained are physical locations that have particular electrical, magnetic, optical, or organic properties corresponding to the data bits.

The data bits may also be maintained on a computer readable medium including magnetic disks, optical disks, organic disks, and any other volatile or non-volatile mass storage system readable by the processing unit. The computer readable medium includes cooperating or interconnected computer readable media, which exist exclusively on the processing system or is distributed among multiple interconnected processing systems that may be local or remote to the processing system.

It should be understood that the programs, processes, methods and systems described herein are not related or limited to any particular type of computer or network system (hardware or software), unless indicated otherwise. Various types of general purpose or specialized computer systems may be used with or perform operations in accordance with the teachings described herein.

In view of the wide variety of embodiments to which the principles of the present invention can be applied, it should be understood that the illustrated embodiments are exemplary only, and should not be taken as limiting the scope of the present invention. For example, the steps of the flow diagrams may be taken in sequences other than those described, and more or fewer elements may be used in the block diagrams. While various elements of the preferred embodiments have been described as being implemented in software, other embodiments in hardware or firmware implementations may alternatively be used, and vice-versa It will be apparent to those of ordinary skill in the art that methods involved in layered aligned polymer structures and methods of making same maybe embodied in a computer program product that includes a computer usable medium. For example, such a computer usable medium can include a readable memory device, such as, a hard drive device, a CD-ROM, a DVD-ROM, or a computer diskette, having computer readable program code segments stored thereon. The computer readable medium can also include a communications or transmission medium, such as, a bus or a communications link, either optical, wired, or wireless having program code segments carried thereon as digital or analog data signals.

The systems and methods of the present invention can be used in a plurality of applications. For example, single layers of aligned collagen can be manufactured which can be used as a test bed for assessing effects of aligned collagen matrices on cellular behavior in connective tissue fibroblasts, in epithelia and in endothelia Further they can generate scaffolding to promote adhesion and proliferation of cell populations in corneal epithelium and/or corneal endothelium.

Further, these embodiments of the present invention can be used to generate multiple layers of aligned collagen which can be used as a test bed for examination of the behavior of cells in anisotropic extracellular matrix including cartilage fibroblasts, corneal keratocytes, and tendon fibroblasts. They can generate connective tissue scaffolding for repair and promotion of cellular adhesion and proliferation that can be used in, for example, but not limited to, artificial corneal replacement, corneal repair material, transfer scaffolding for epithelial transplants, transfer scaffolding for endothelial transplants, tendon replacement or repair, ligament replacement or repair and annulus fibrosis replacement or repair. In addition, biocompatible strengtheners for natural and/or artificial materials for use in tissue repair or replacement can be generated using the embodiments of the present invention. These can be rolled up to perform annulus fibrosis function while embedded in a poly(vinyl alcohol) matrix, or be used as strengtheners for corneas made from artificial materials, for example, poly HEMA and/or be a resorbable anchor for tissue repair.

Further applications of the preferred embodiments include generating multiple layers of other aligned biopolymers that can be used as biocompatible scaffolding, for example, “braids” to strengthen stents, or other implants. They can also be used as guidance for nerves, for example, nerve cuffs. Other applications having layers where alignment is not important that can benefit from the layered polymer structures of the present invention include support for species embedded in multiple layers such as cells and drug release applications.

In addition, applications that include non-biopolymers may benefit from the deposition of layered, aligned polymer structures, for example, generation of optical storage media.

### EXAMPLE 6

The physical principles governing spin-coating flows are discussed in further detail below. Spin-coating is one of the simplest and most common approaches for applying thin films to wafers and substrates. Thus, the behavior of fluids in this kind of configuration has been well studied. In its simplest form the fluid is undergoing quasi-solid body rotation on the disk and the only velocity components are rotational and radial. The fluid behavior is therefore controlled by the balance between the centripetal and viscous forces.

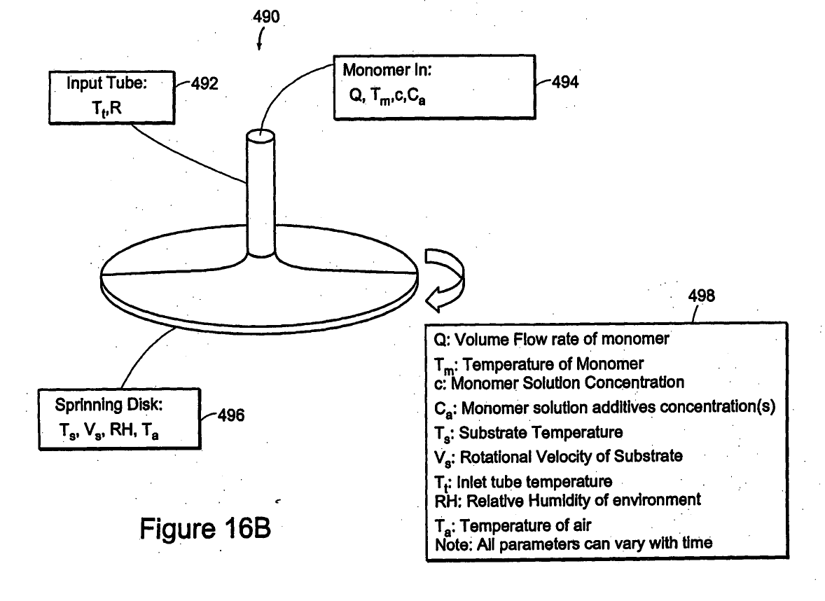

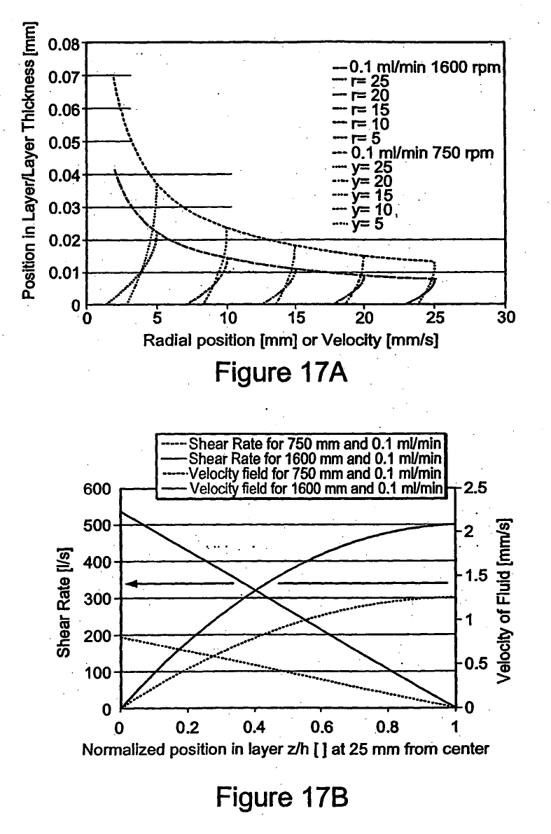

For the situation where there is a steady flow rate of fluid onto a spinning disk the solution requires an analysis of the full Navier-Stokes equation. Leneweit et al. (G. Leneweit, K. G. Roesner, R. Koehler, Experiments in Fluids 26, 75-85 (1999)) outlined work by Rauscher et al. (J. W. Rauscher, R. E. Kelly, J. D. Cole, Journal of Applied Mechanics 40, 43-47 (1973)) that generates a deceptively simple solution to the steady flow problem. To first order, this solution is:

$$u = \frac{r {ω}^{2} {h}^{2}}{v} ( \frac{z}{h} - \frac{1}{2} {( \frac{z}{h} )}^{2} ) ; h = {( \frac{3}{2 π} \frac{Qv}{{ω}^{2} {r}^{2}} )}^{1 / 3} \qquad Equation 3$$

where u is the radial velocity as a function of z away from the disk at a position r radially, h is the thickness of the film, w the rotation rate of the disk, v is the kinematic viscosity of the solution and Q the volume flow rate. Calculations made using these relationships for practical conditions are summarized in FIGS. 17A and 17B.

FIG. 17A is a graphical representation 500 of the relationships between position within the layer or layer thickness on the ordinate and radial position or velocity on the abscissa for conditions of 750 rpm (dashed lines) and 1600 rpm (continuous lines) with constant addition of collagen solution (0.1 ml/min) to the center of a 2.5 cm radius disk. For both sets of flow conditions, the fluid layers are of the order of 10 μm at 25 mm from the point of deposition. In addition, from 10 mm to 25 mm neither layer thickness varies by more than 10 μm. FIG. 17B is a graphical representation 520 of the relationships between shear rate or velocity of fluid ordinate and normalized position in layer at 25 mm from center on the abscissa for conditions of 750 rpm (dashed lines) and 1600 rpm (continuous lines). Spin coating flow velocity profiles are semi-parabolic with high shear rates at the disk surface (200-550 s⁻¹) and moderate flow rates at the free surface (1.25-2 mm/sec). This analysis predicts that a virtually flat layer can be generated that will confine the growing collagen film and provide shear rates in excess of 100 s⁻¹ near the substrate surface. These shear rates compare well with those seen experimentally in the work with DNA. Another critical parameter, the relaxation time for the growing collagen fibril, must vary with length of the growing fibril.

Orthogonal stacking of multiple layers of aligned collagen fibrils was produced by placing a glass substrate off-center in a specially designed offset disk holder (FIG. 8A). Collagen solution was applied at the center of rotation for the entire assembly as shown in FIG. 8A. After one layer of collagen was applied, the glass substrate could be rotated any arbitrary angle and another layer was applied At the exact center of the glass substrate, the collagen layers cross each other at the same angle by which the glass substrate was rotated.

The surface profiles of the films generated are relatively flat to within 10 μm over the distance of 1.0 cm at the outer edge of the disk. At 2.5 cm from the center, the film thickness is between 8.0 and 12.0 μm for the two flow regimes examined. Briefly, cold collagen solution was applied at 0.1 ml/min for 60 minutes to a warmed (37° C.), centered 5 cm diameter glass disk, cleaned for 1 hour in Micro90™ solution. To make isotonic, neutralized solutions of bovine collagen, Vitrogen™ (Cohesion Technologies, BC) collagen solution, chilled to 4-6° C. is mixed into 10×PBS in an 8:1 ratio. One part 0.1 M NaOH is then added. The pH of the solution is adjusted to 7.4 by the addition of 0.1 M HCl or 0.1M NaOH. The neutralized isotonic collagen solution is stored at 4-6° C. for a maximum of 4 hours before use. If necessary, to increase the wetting ability of the collagen solution, Triton X100 surfactant (Sigma Aldrich, Mo.) is added (about 1 drop/20 ml collagen solution).

In a preferred embodiment, collagen films are made in a spin-coater (WS-400A-6NPP/LITE, Laurell Technologies) where the internal environment can be controlled to 25° C. and 100% RH. Temperature control is suitably accomplished by addition of a PELT-1 and PTC-1 temperature controller (Sable Systems, NV) to the spin coater environmental chamber. Suitably, humidity can be controlled by attaching a DG-1 humidity controller (Sable Systems, Nev.) to the spin coater environmental chamber. Preferably collagen films are made, and solutions and substrates prepared, in a class 100 laminar flow clean hood (Air Science Technologies, Fla.) to prevent contamination by dust or other particulates.

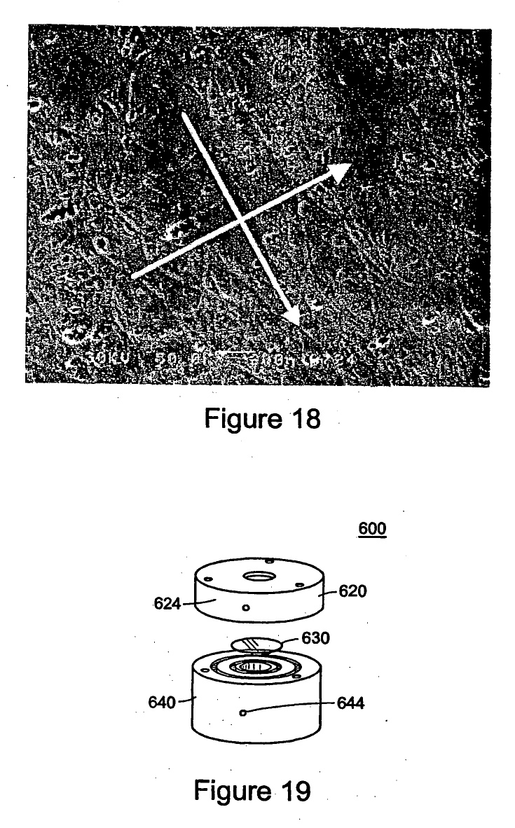

The spin rate was 750 rpm and the chamber humidity was greater than RH=95%. Following the coating process the film was allowed to dry and then prepared for SEM. FIG. 18 shows a SEM image 540 of fibril alignment 2.0 cm from the fluid deposition point; the flow rate was 0.25 ml/min and the rotation velocity was 1600 rpm. The top layer in this particular area formed incompletely due to de-wetting during the film formation, and the lower, orthogonal layer of fibrils can be seen passing under the top layer.

In general, improving wet-ability and preventing de-wetting can be accomplished by methods known in the art. Glass surfaces can be treated by hydrosilation (J. J. Pesek, M. T. Matyska, Interface Science 1997 5: 103-117). Hydrosilation is a robust approach that is slightly more complex than traditional silanization (J. J. Pesek, et al., Journal of Chromatography A 1998 818: 145-154). The natural hydroxide (Si—OH) group of the glass surface is silanized using a reactive silane agent. This surface is then reacted in the presence of a catalyst such as hexachloroplatinic acid and a CH2=CH—R molecule which allows a vast array of potential surface treatments. The effect of a given treatment can be assessed by the contact angle which can be determined using optical methods known in the art. See, for example, D. J. Shaw, Colloid and Surface Chemistry (Butterworth-Heineman, Oxford, ed. Fourth, 1994). Exemplary treatments include those that can produce a contact angle <30 (“hydrophilic”—e.g. Allyl glycidyl ether), those that can produce a contact angle >120 (“hydrophobic”—e.g. 1-octene) and those that produce steric protection (e.g. poly (ethylene oxide) based systems).

In some embodiments, the formed collagen films can be further modified with additives to promote cell attachment and proliferation, as noted above. Such additives may include proteoglycans, laminin, fibronectin, vinculin or integrin moieties and mixtures thereof. Where the additive is a proteoglycan, the proteoglycan is suitably selected from the group consisting of decorin, lumican, biglycan, keratocan or syndican.

In some embodiments, the proteoglycan is decorin Decorin represents nearly 40% of the total proteoglycan in the cornea and has been proposed to play an important role in fibrillogenesis, tissue repair and the regulation of transforming growth factor-beta (TGF-β has a single chondroitin/dermatan sulfate chain near the N-terminus, and is subject to N-linked glycosylation). Decorin can bind to collagens, TGF-β, epidermal growth factor (EGF) receptors and fibronectin. In decorin-null mice, the collagen network in the skin is shown to be loosely packed with irregular contours and unnatural lateral fusion of collagen fibrils is evident. Decorin core protein can be purified from tissue in a technique that preserves native structure and function (C. T. Brown et al., Protein Expr Purif 25, 389-99 (August 2002). Unlike most procedures this method does not rely on the use of strong denaturing reagents that may compromise biological activity. For example, exhaustive extraction of 100 pulverized bovine corneal stromas (99.7 g wet) yielded 225 mg of total GAG, 2.45 g of total protein and 110.8 mg total decorin. The final purification yielded 35 mg of decorin core protein per 100 corneas. This extraction consisted of a heterogeneous mixture of proteins and proteoglycans (CSPGs and KSPGs). Elution of the proteoglycans with 1.5 M NaCl resulted in 98% recovery of GAGs and 95% recovery of the decorin. The presence of decorin during collagen fibrillogenesis prevents the lateral fusion of fibrils, which is apparently responsible for the reduction in the rate of fibrillogenesis reported in the literature. When used, decorin is present in the collagen monomer solution at the concentration of about 1 to about 100 μg/ml, suitably about 5 to about 50 μg/ml.

In some embodiments, the proteoglycan is lumican. It is known that mice homozygous for a null mutation in lumican (a keratan sulfate proteoglycan) do not have clear corneas. X-ray diffraction studies indicate that in these corneas the collagen network is in disarray and that there is a significant polydispersity in the diameter of the corneal collagen fibrils that may lead to the observed opacity.

### EXAMPLE 7

As another preferred embodiment of the material described in this invention, epithelial cells and endothelial cell layers are established in the structure generated in either of the first three examples with collagen to develop a corneal construct This construct produces a cornea that is similar physiologically, ultrastructally and biochemically to normal mammalian corneas. The construct is suitable as a physiological or biomechanical model, and may find utility as a material for transplant into human subjects.

Tissue engineering has met with great success in producing artificial skin and is advancing rapidly in the development of artificial vessels. The cornea, being a fairly simple, avascular tissue, is an attractive next milestone for such an approach. Indeed, artificial corneal constructs have been produced already by culturing corneal cells onto and within a collagen gel. Unfortunately, these constructs are not yet suitable for use in the clinic because the primary functions of the cornea (protection, refraction and transmission) are fundamentally dependent on the ultrastructural organization of the stromal matrix. A stromal-centric approach is necessary. Tissue engineering alone has not been able to reproduce the stroma on the nanoscale because, to date, corneal fibroblasts cannot be induced to synthesize normal stromal architecture, nor can it be produced artificially, de novo. However, recent data show that activated human corneal keratocytes can produce the appropriate matrix components, but that they are not organized. Stromal development is a complex event that depends on an intricate choreography of signaling, synthesis and contact guidance. In short, the cells need an organized matrix to direct the synthesis of additional extracellular matrix (referred to herein as “templating”). In preferred embodiment, biomimetic “primary” stromal extracellular matrix (ECM) template provides adequate contact guidance to induce activated stromal keratocytes to produce organized “secondary stroma”. The present invention provides such a “primary” stromal template, de novo, by precisely controlling collagen fibril alignment on the nanoscale using microfluidics and collagen type I self-assembly as described in Examples 1-6 above. Such a primary stroma can be generated either by orthogonally stacking these de novo manufactured lamellae, or by using a small number of them to “contact guide” human corneal keratocytes that have been induced to produce ECM.

Multilaminar collagen structures of the present invention can be used as nanostructure artificial templates (NAI) that are seeded with cells, such as fibroblasts, epithelial cells and endothelial cells to form artificial corneas. In preferred embodiments, artificially produced nanostructured biomaterials are used as a template to induce the replication of corneal ultrastructure by fibroblast cells. In some embodiments, the cells are applied to unstretched NATs. In other embodiments, the cells are applied to NATs that are stretched to mimic physical environment of the cornea in situ. The NAT can be simply constructed from collagen, or may also include additives such as proteoglycans, laminin, fibronectin, vinculin, integrin moieties or mixtures thereof Where the additive is a proteoglycan, the proteoglycan is suitably selected from the group consisting of decorin, lumican, biglycan, keratocan, syndican or mixtures thereof Nanostructured materials derived from this process can be used to construct an artificial corneal stroma for use in replacement or repair of corneal tissue. This templating approach requires the availability of an appropriate nanostructured artificial template (NAT) such as that described in Examples 1-6 above. In preferred embodiments, the NAT is a nanostructured artificial collagen template.

In one embodiment, the present invention provides a method comprising the steps of providing a nanostructured artificial template as a primary stroma; contacting the primary stroma with a first population of cells; and activating the first population of cells to form extracellular cellular matrix material on the nanostructured artificial template to produce a secondary stroma, thereby producing a populated biomimetic corneal stroma In some embodiments, the method further comprises the step of contacting a first surface of the populated biomimetic corneal stroma with corneal epithelial cells. In some embodiments, the method further comprises the step of contacting a second surface of the populated biomimetic corneal stroma with corneal endothelial cells.

In the adult cornea, the stromal keratocytes exist in a quiescent state with a very low rate of cell proliferation (Zieske, J. D., et al., Kinetics of keratocyte proliferation in response to epithelial debridement. Exp Eye Res, 2001. 72(1):33-9). Upon corneal injury, the keratocytes are stimulated to proliferate and migrate to the wound site. The activated keratocytes are termed fibroblasts. In some types of wounds, the fibroblasts differentiate further into myofibroblasts and exhibit filaments consisting of a-smooth muscle actin (Jester, J. V., et al., Expression of alpha-smooth muscle (alpha-SM) actin during corneal stromal wound healing. Invest Ophthalmol Vis Sci 1995. 36(5):809-19; Masur, S. K., et al., Myofibroblasts differentiate from fibroblasts when plated at low density. Proc Natl Acad Sci U S A, 1996. 93(9):4219-23). The fibroblasts and myofibroblasts also synthesize and secrete type I and III collagens, which are associated with scar formation (Nusgens, B. V., et al., Topically applied vitamin C enhances the mRNA level of collagens I and III, their processing enzymes and tissue inhibitor of matrix metalloproteinase 1 in the human dermis. J Invest Dermatol, 2001. 116(6):853-9; Ohgoda, O., et al., Fibroblast-migration in a wound model of ascorbic acid-supplemented three-dimensional culture system: the effects of cytokines and malotilate, a new wound healing stimulant, on cell-migration. J Dermatol Sci, 1998. 17(2):123-31; Appling, W. D., et al., Synergistic enhancement of type I and m collagen production in cultured fibroblasts by transforming growth factor-beta and ascorbate. FEBS Lett, 1989. 250(2):541-4). These cells also secrete a variety of other ECM components.

When keratocytes are isolated and placed into culture in the presence of serum or growth factors, they become proliferative and are most properly termed fibroblasts. When these cells are grown in culture, they become increasingly quiescent as they reach confluence. However, in the early 1980's, several groups found that the addition of ascorbic acid (vitamin C) increased the proliferative rate of cultured fibroblasts (Tajima, S. and S. R. Pinnell, Regulation of collagen synthesis by ascorbic acid. Ascorbic acid increases type I procollagen mRNA. Biochem Biophys Res Commun, 1982. 106(2):632-7; Lyons, B. L. and R. I. Schwarz, Ascorbate stimulation of PAT cells causes an increase in transcription rates and a decrease in degradation rates of procollagen mRNA. Nucleic Acids Res, 1984. 12(5):2569-79.) In addition, it was reported that ascorbic acid stimulated the synthesis and secretion of ECM components such as type I and type m collagens (Nusgens, B. V., et al., 2001; Appling, W. D., et al., 1989; Hata, R. and H. Senoo, L-ascorbic acid 2-phosphate stimulates collagen accumulation, cell proliferation, and formation of a three-dimensional tissuelike substance by skin fibroblasts. J Cell Physiol, 1989. 138(1):8-16; Saika, S., et al., L-ascorbic acid 2-phosphate, a phosphate derivative of L-ascorbic acid, enhances the growth of cultured rabbit keratocytes. Graefes Arch Clin Exp Ophthalmol, 1991. 229(1): 79-83). Ascorbic acid acts as a cofactor for the enzymes responsible for hydroxylation of the lysine and proline residues on procollagen. These hydroxylations are required for the proper assembly of procollagen, which is secreted into the ECM. Subsequently, it was found that a more stable form of ascorbic acid, L-ascorbic acid 2-phosphate, has a far more potent effect on synthesis and secretion of ECM materials. L-ascorbic acid 2-phosphate was also found to stimulate the stratification of several fibroblast types, including dermal and corneal fibroblasts. This method of allowing the fibroblasts to assemble their own matrix has been used to engineer tissues including skin (Germain, L., et al., Can we produce a human corneal equivalent by tissue engineering? Prog Retin Eye Res, 2000. 19(5):497-527; Michel, M., et al., Characterization of a new tissue-engineered human slin equivalent with hair. In Vitro Cell Dev Biol Anim, 1999. 35(6):318-26) and blood vessels (Germain, et al., 2000; Gennain, L. M. et al., Tissue engineering of the vascular system: from capillaries to larger blood vessels. Med Biol Eng Comput, 2000. 38(2):232-40). It has been reported that a dermal substitute was reconstructed entirely from cells grown in the presence of L-ascorbic acid 2-phosphate and including the addition of endothelial cells to generate capillary-like structures (Guido, S. and R. T. Tranquillo, Methodology for the systematic and quantitative study of cell contact guidance in oriented collagen gels. Journal of Cell Science, 1993. 105:317-331). In the blood vessel model, the fibroblasts assembled a dense ECM containing collagens, glycosaminoglycans and elastin.

There is evidence that substrate surface structure can control cultured fibroblast morphology (Guido, S. and R. T. Tranquillo, Methodology for the systematic and quantitative study of cell contact guidance in oriented collagen gels. Journal of Cell Science, 1993. 105: 317-33), which in turn may control the structure of secreted matrix in culture. (Wang, J. H., et al., Cell orientation determines the alignment of cell-produced collagenous matrix. J Biomech, 2003. 36(1): 97-102). This effect has been termed “contact guidance”. In aligned collagen matrices, it has been reported that the depth of neurite elongation into the aligned media is far greater than for random matrix controls (Dubey, N., et al., Guided neurite elongation and Schwann cell invasion into magnetically aligned collagen in simulated peripheral nerve regeneration. Exp Neurol, 1999. 158(2): 338-50). It has also been reported that nerve generation occurs more rapidly if axons are induced to traverse aligned collagen matrices rather than random collagen controls (Verdu, E., et al., Alignment of collagen and laminin-containing gels improve nerve regeneration within silicone tubes. Restor Neurol Neurosci, 2002. 20(5): p. 169-79).

In addition to contact guidance, fibroblast and smooth muscle cell morphology, motion and differentiation may be influenced by strain or stretch of the substrate (Wang, J. H. and E. S. Grood, The strain magnitude and contact guidance determine orientation response of fibroblasts to cyclic substrate strains. Connect Tissue Res, 2000. 41(1): 29-36; Girton, T. S., et al., Confined compression of a tissue-equivalent: collagen fibril and cell alignment in response to anisotropic strain. J Biomech Eng, 2002. 124(5): 568-75; Altman, G. H., et al., Cell differentiation by mechanical stress. FASEB J, 2002. 16(2): p. 270-2; Altman, G. H., et al., Advanced bioreactor with controlled application of multi-dimensional stain for tissue engineering. J Biomech Eng, 2002. 124(6): 742-9).

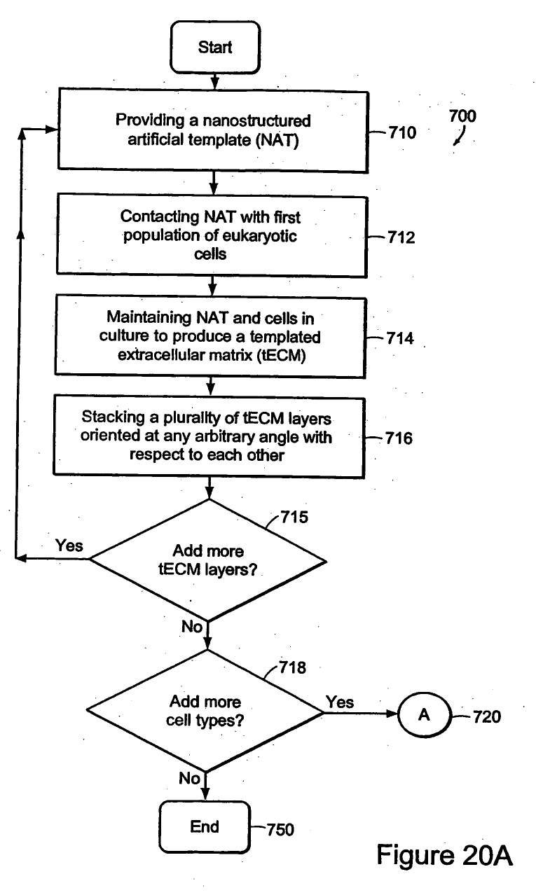

Multilaminar aligned collagen structures are formed as described above and used as nanostructure artificial templates (NAT). In some embodiments, the multilaminar aligned collagen structures further comprise additives, such as proteoglycans. A preferred proteoglycan is decorin. In some embodiments, the nanostructure artificial templates are seeded with cells in a stress-free state. In other embodiments, strain is applied to the nanostructure artificial template before being seeded with cells. FIG. 19 schematically illustrates an Ussing-style perfusion chamber and a nanostructured artificial template (NAT) comprising collagen placed in between the half-chambers and a pressure is applied to apply strain to the structure. The perfusion chamber 600 has an upper portion 620 and a lower portion 640 that enclose the nanostructure artificial template 630. The upper portion 620 and a lower portion 640 are provided with respective perfusion ports 624 and 644. Fluids can be applied to the perfusion ports, and a pressure differential can apply stress to the nanostructure artificial template 630, causing it to deform into, preferably, a curved shape.

In preferred embodiments, the nanostructured artificial collagen template is seeded with fibroblasts and maintained in culture for a period before placing it in the Ussing-style perfusion chamber. In preferred embodiments, the pressure placed on the nanostructured artificial collagen template is selected to produce strains similar to strains on actual in vivo corneal lamellae. In other embodiments, fibroblasts can be applied to a pre-strained template.

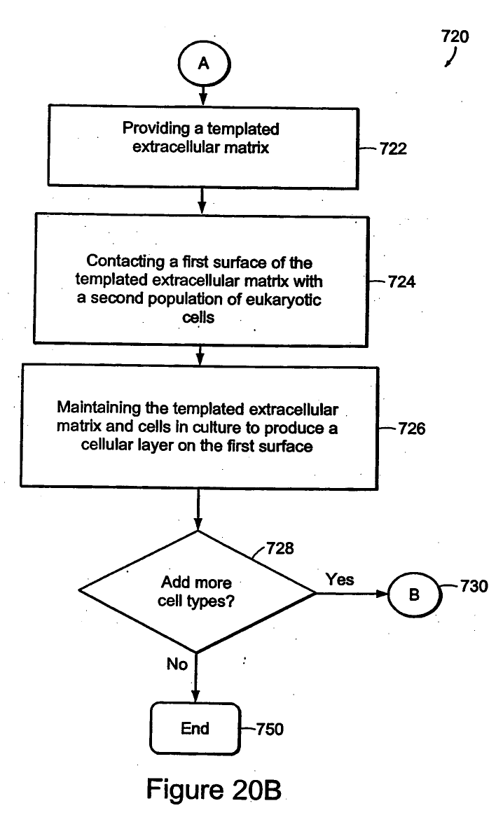

FIGS. 20A-20C illustrate a flow chart for producing a cellular biomimetic stroma from a nanostructured artificial template in accordance with a preferred embodiment of the present invention. With reference to FIG. 20A, the method 700 includes the step 710 of providing a nanostructured artificial template (NAT). In general the nanostructured artificial template is an aligned array of linear structures sufficient to induce a fibroblast to produce an aligned extracellular matrix which can in turn iteratively act to induce fibroblasts to produce subsequent aligned extracellular matrix. In preferred embodiments, the nanostructured artificial template is a multilaminar aligned collagen structure formed as described above. Alternatively, a nanostructured artificial template can be constructed from other non-toxic materials such as non-collagen polymers, glass, metals or ceramics that can be structured into a linear array by photolithography, etching, molecular growth or other means suitable to produce a pattern of the required scale.

The method 700 also includes the step 712 of contacting the NAT with a first population of cells capable of iteratively producing an organized extracellular matrix array in response to the nanostructured artificial template. Suitable cells are eukaryotic cells. In a preferred embodiment, the eukaryotic cells are fibroblasts. In particularly preferred embodiments, the nanostructured artificial template comprises collagen and the eukaryatic cells are mammalian fibroblasts. In some embodiments, the mammalian fibroblasts are derived from stimulated corneal stromal keratocytes.

In one preferred embodiment, stromal keratocytes harvested from human corneal donors are stimulated to produce extracellular matrix (ECM) using vitamin C (ascorbic acid) or a more stable derivative of vitamin C (Lascorbic acid 2-phosphate), pharmaceutically acceptable organic and inorganic acid addition salts thereof preferably sodium ascorbyl phosphate or magnesium ascorbyl phosphate and cultured onto the surface of the nanostructured artificial collagen template. The culture with the stimulated fibroblastic cells is maintained for a period of time and then the activity of the corneal fibroblasts is arrested by replacing the vitamin C in the perfusate with fetal bovine serum, thereby arresting the fibroblastic activity of the cells and restoring them to their quiescent keratocyte phenotype. This procedure can produce a three-dimensional extracellular matrix that reflects and incorporates the structure of the nanostructured artificial collagen template. Cellular secretion of extracellular matrix (ECM) in the nanostructured artificial collagen template produces a “templated ECM” (tECM). Depending on the amount of organized matrix produced by the fibroblasts, the tECM may be used alone or may be stacked onto other such constructs and then used in the repair or replacement of diseased or damaged corneal stromal tissue. In addition, single or multiple tECMs may be used as a substrate onto which corneal epithelium and/or endothelium may be cultured to produce a functional artificial cornea.

Cell harvesting procedure. Corneal buttons are removed from the central cornea using an 8-mm trephine. The corneal buttons are placed in Dulbecco's minimal essential medium (DMEM) plus antibiotics and cut into quarters. The quartered buttons are then placed in 21 ml of DMEM containing 3.3mg/ml collagenase (type L.C8170; Sigma Aldrich, Mo.) and incubated at 37° C. with shaking for 30 min. The tissue plus solution is then vortexed for 30 seconds and the tissue removed using a cell strainer. Collagenase digestion is repeated for 60 minutes, the tissue strained, and then a third repeat of 180 minutes. The cells in each of the three collagenase digestions are be collected by low speed centrigation and resuspended in DMEM. Our studies have shown that the cells from the third digestion represent the purest population of keratocytes. Cell numbers are determined using a hemocytometer and the cells are resuspended in DMEM containing 1% platelet-poor horse serum (Sigma Aldrich, Mo.) and plated in uncoated culture dishes. Culture medium is changed every 2-3 days.

The method 700 also includes the step 714 of maintaining the NAT with a first population of cells in culture to produce a templated extracellular matrix.

Culturing cells onto NAT. In a preferred embodiment, nanostructured artificial collagen templates in PBS are placed onto the polycarbonate membrane (Costar, Charlotte, N.C.) of a 12 mm diameter Transwell™ insert (for unstretched protocol) or clamped into the pressurized perfusion chamber (for stretched protocol). The stromal lamellae are allowed to equilibrate and settle overnight prior to seeding. Human stromal fibroblasts (2×10) are added to the Transwell™ in DMEM containing 10% FBS and 1 mM L-ascorbic acid 2-phosphate. The lamellae and fibroblasts are incubated at 37° C. and medium is changed every other day. For controls, the Transwell™ membrane alone is the substrate for the cell culture. The process of creating one layer of secreted matrix is extended to generate a full-thickness stroma (450 μm). The number of stromal templates, n, is chosen such that the final thickness of the secreted matrix alone multiplied by n equals ˜450 μm. The mounted stromal templates is seeded with activated human keratocytes covered with a second template matrix and cultured until they have generated the anticipated amount of stromal tissue. At this time, the perfusion solution is switched to arrest matrix production and to promote the differentiation to quiescent keratocytes. The constructs are removed from their mounts and, if possible, the templates are dissected away. The remaining secreted stromal matrices are stacked to generate a thick stromal construct. The constructs are transferred to perfusion chambers and loaded (stretched) for a period of up to 8 weeks. The stacks of de novo lamellae are populated with keratocytes to generate an appropriate stromal analog. Unlike stromas made via templating, the keratocytes in this construct are not activated to produce matrix, and express the quiescent keratocyte phenotype. The following seeding method is used to facilitate population of the de novo matrix with keratocytes,. Following the generation of thick stroma, the construct is placed onto a Transwell™ culture dish and pinned down lightly with a 1.0 cm 0-ring (allowing the outer edges of the construct to swell and fray). Isolated human keratocytes and medium are added to the Transwell™ culture dish and allowed to infiltrate the edges of the construct for 24 hours. At the end of that time, a 1.4 cm O-ring is used to clamp the edges of the construct and the 1.0 cm O-ring will be removed. Further infiltration of the construct by the keratocytes can be facilitated by maintaining the construct in this configuration until keratocytes reach the center (as determined by optical microscopy). Following infiltration of the keratocytes, the construct is transferred to the perfusion chamber and loaded (stretched) for a period of up to eight weeks. Confocal microscopy is used to assess the infiltration of the keratocytes into the center of the stromal construct. The resulting cultured tissue can be harvested at 1, 4 and 8 weeks for light microscopy, TEM, QFDE and immunohistochemistry. A successful construct contains a stable population of quiescent keratocytes with few fibroblasts or myofibroblasts. The cells should exhibit an elongated flat morphology and reside between adjacent lamellae. The construct ultrastructure should comprise multiple layers of aligned type I collagen with decorin distributed, evenly throughout. Fibroblasts are found to populate the nanostructured artificial template and to secrete additional extracellular matrix.

The method 700 also includes the step 718 of deciding whether an additional population of cells should be added to the templated extracellular matrix. If the answer is “no”, the process is ended 750. Otherwise, the method 700 is continued in method 720.

With reference to FIG. 20B, the method 720 includes the step 722 of providing a templated extracellular matrix, such as that produced by step 714. Method 720 also includes the step 724 of contacting a first surface of the templated extracellular matrix with a second population of cells. Suitable cells are eukaryotic cells.

In preferred embodiments, the second population of cells is a population of mammalian corneal epithelial cells. The corneal epithelium is a multicellular “tight” stratified squamous epithelium comprising three distinct functional layers. It is approximately 50 microns thick in humans. The deepest cellular layer is the stratum germinatum, the only layer capable of undergoing mitosis. The middle layer comprises the daughter cells (wing cells) of the basal layer which are pushed anteriorly. The surface layer comprises the squamous cells that form the complete tight junctions which generate the primary barrier to transport in the cornea and are critical to understanding its vegetative physiology. As the leading edge of the tough ocular tunic, the epithelial cells serve to protect and defend the underlying corneal stromal tissue. In constant state of turnover, the epithelial cells continually renew the corneal surface. The complete tight junctions, maintained during the turnover process, limit fluid transport and prevent infection Active chloride transporters give the epithelium a limited ability to move fluid out of the corneal to the tear film. However, physiologically, the epithelium is primarily a barrier. Following injury epithelial cells rapidly re-cover the affected area and restore corneal clarity. It is becoming clear that interaction between the epithelial cells and underlying stromal cells is necessary to fully respond to injury.

The templated extracellular matrix may be stressed or unstressed. In preferred embodiments, the templated extracellular matrix is stressed into a curved form, and the first surface is the convex face of the curved templated extracellular matrix.

The method 720 also includes the step 726 of maintaining the templated extracellular matrix with a second population of cells in culture to produce a templated extracellular matrix having a layer of the second population of cells on the first surface.

In one embodiment, the second population of cells is added to the multilaminar templated extracellular matrix as follows. The methodology for the isolation and cultivation of human corneal epithelial cells is known in the art. In brief, the limbal ring of the cornea is incubated 18-24 hours in a dispase solution at 2-8° C. The epithelium is separated and treated briefly with trypsin-EDTA (Life Technologies, Inc, MD) solution to dissociate the cells. The trypsin action is stopped by the addition of Trypsin Neutralizing Solution (Clonetics, MD); the cells are pelleted, resuspended in Keratinocyte-SFM medium (Life Technologies, Inc.), and seeded onto T-75 tissue culture flasks coated with fibronectin-collagen (FNC) coating mix (Biological Research, MD). We routinely obtain one to two T-75 flasks of cells per cornea using this methodology (2×10⁶ cells/T-75 flask). The limbal rings are from tissue used for corneal transplantation that would otherwise be discarded. Human corneas deemed unsatisfactory for transplant can also be obtained from NDRL.

A corneal epithelium cultured atop a corneal keratocyte-assembled templated extracellular matrix can be obtained as follows. After stabilization of the stromal construct, the media is removed from the construct surface and the construct is coated with FNC for one minute to promote adhesion of epithelial cells. The FNC is then removed and a suspension of corneal epithelial cells (2×10⁵/well) is added to the coated construct. After the epithelial cells are seeded onto the matrix, Keratinocyte-SFM media is added to the anterior side of the construct and it is incubated at 37° C. After 24 hours, medium is added to the posterior side of the construct as needed. The cultures are maintained for an additional 24 hours (two days total), after which all media is removed. Fresh Keratinocyte-SFM medium supplemented with 0.3% FBS and 1.7 mM Ca++ (1.73 mM final concentration) is added to the posterior side only, thus causing the epithelium to be air lifted and maintained with a minimal covering of media. Calcium and FBS are added to stimulate epithelial stratification and differentiation. The cultures are maintained for one to two weeks, with media being changed three times per week.

The method 720 also includes the step 728 of deciding whether an additional population of cells should be added to the templated extracellular matrix having a layer of the second population of cells on the first surface. If the answer is “no”, the process is ended 750. Otherwise, the method 720 is continued in method 730.

With reference to FIG. 20C, the method 730 includes the step 732 of providing a templated extracellular matrix having a layer of the second population of cells on the first surface, such as that produced by step 726. Method 730 also includes the step 734 of contacting the second surface of the templated extracellular matrix with a third population of cells. Suitable cells are eukaryotic cells.

In preferred embodiments, the third population of cells is a population of mammalian corneal endothelial cells. Corneal endothelium is a single layer of tissue that forms a boundary between the corneal stroma and the anterior chamber. The endothelium from young individuals consists of polygonal-shaped cells, 4-6 mm thick with a diameter of around 20 mm. Corneal endothelial cells express occluding an integral membrane protein associated with tight junctions, and ZO-1, a member of a submembranous cytoplasmic complex associated with tight junctions. Abundant mitochondria indicate the high metabolic activity of these cells and are consistent with their role as the major fluid transporting layer in the corneal. The basal (anterior-most) aspect of corneal endothelial cells rests on Descemet's membrane, the thick basement membrane that is secreted by the endothelium. Proteins, such as vinculin, talin, b3-integrin, and alpha-v, beta-5 integrin are expressed in corneal endothelial cells, suggesting that they form structures that facilitate normal cell-substrate adhesion. The primary function of the endothelium is to maintain corneal transparency by regulating corneal hydration against a large swelling pressure. Proteoglycans associated with stromal collagens bind water and produce a pressure gradient across the endothelium. The endothelium counteracts the tendency of the corneal stroma to swell by removing excess stromal fluid via the activity of Na+/K+-ATPase and bicarbonate-dependent Mg2+-ATPase.

The templated extracellular matrix may be stressed or unstressed. In preferred embodiments, the templated extracellular matrix is stressed into a curved form, and the second surface is the concave face of the curved templated extracellular matrix.

The method 730 also includes the step 736 of maintaining the templated extracellular matrix with a third population of cells in culture to produce a templated extracellular matrix having a layer of the third population of cells on the second surface. Methods of human corneal endothelial cell culture are disclosed in U.S. Pat. No. 6,548,059, which is hereby incorporated by reference in its entirety. In preferred embodiments, donor corneas stored in Optisol-GS at 4° C. are obtained from NDRL For culturing of endothelial cells, corneas are placed in a petri dish containing Medium 199 and 50 μg/ml gentamicin. Under a dissecting microscope, Descemet's membrane with the attached endothelium is stripped from the stroma and placed in a 15 ml centrifuge tube containing 0.2 mg/ml EDTA in Hank's BSS, pH 7.4. The tissue is incubated for 1 hour at 37° C., then cells are detached from

Descemet's membrane by vigorous disruption with a flame-polished pipette. Cells are pelleted and then re-suspended in culture medium containing OPTIMEM-1 culture medium supplemented with 8% FBS, 40 ng/ml FGF, 5 ng/ml EGF, 20 ng/ml NGF, 20 μg/ml ascorbic acid, 0.005% human lipids, 200 mg/L calcium chloride, 0.08% chondroitin sulfate, 1% RPMI-1640 multiple vitamin solution, 50 μg/ml gentamicin, and antibiotic/antimycotic solution (1/100). Cells are incubated in six-well tissue culture plates at 37° C. in 5% CO2, and medium is changed every other day. With this methodology, cells reach confluence in 10 to 14 days, after which time they will be subcultured and seeded at a split ratio of 1:4 or 1:8. Only cells from primary culture or passage one are used. Stromal matrix constructs (multilaminar templated extracellular matrix) are washed with growth medium prior to seeding cells directly onto the matrix. The stromal surface can also be pre-coated with fibronectin, laminin, or type IV collagen alone or in combination prior to cell seeding to facilitate endothelial cell adhesion. The cell-matrix complex is then incubated, endothelial side-up, at 37 degrees Celsius in a 5% CO₂, humidified chamber for various periods of time.

The method 730 also includes the step 738 of deciding whether an additional population of cells should be added to the templated extracellular matrix now having a layer of the second population of cells on the first surface and a layer of the third population of cells on the second surface. If the answer is “no”, the process is ended 750. Otherwise, the method 730 is continued by repeating method 720 or method 730 as needed.

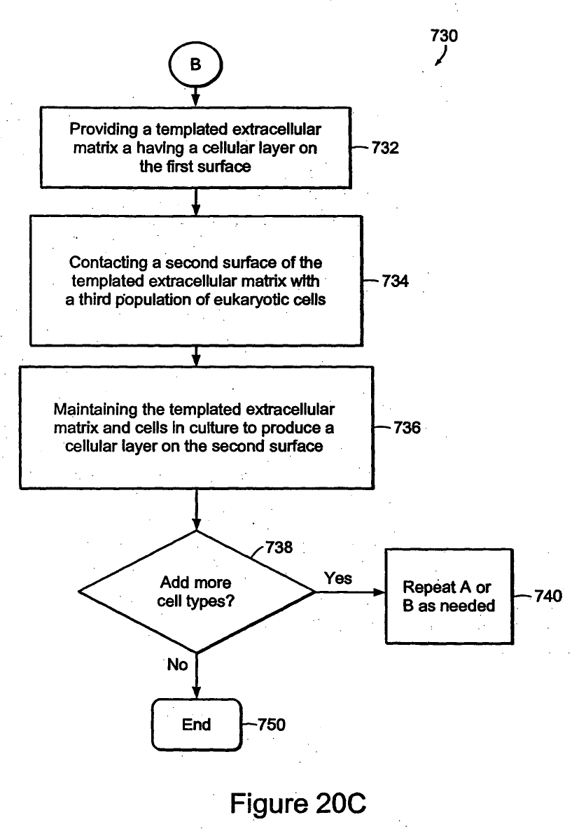

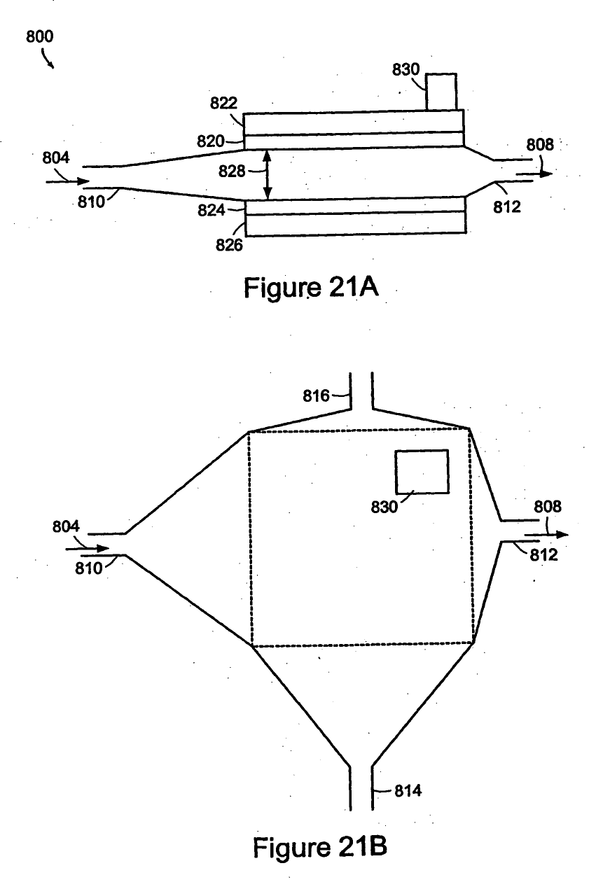

FIG. 21A is a schematic diagram illustrating a flow chamber 800 for generating layered aligned polymer in accordance with a preferred embodiment of present invention. A preferred embodiment provides a method to generate single or multiple layers of aligned polymer fibrils by introducing monomer solution between two surfaces, with an adjustable gap between them The flow chamber 800 is lined by a collagen accepting layer 822 and a collagen rejecting layer 820 that, together with inlet 810 and outlet 812, define a space in which collagen polymerizes. The collagen rejecting surface 820 is attached to an upper thermoelectric device 822; the collagen accepting surface 822 is attached to a lower thermoelectric device 820. The upper and lower thermoelectric devices are preferably provided with heat sinks that may be solid or that may contain channels through which cooling fluids may be circulated. The height 828 is adjustable using a micromanipulator 830 that is attached to the upper thermoelectric device 822.

A collagen accepting surface 822 may be generated by coating the surface may be generated by coating the surface with antibodies to collagen, by plasma cleaning, by cleaning with Micro90™, by functionalization, or by treating the surface with any methods known in the art to attract and promote adherence of collagen monomer or polymer units. A collagen rejecting surface 820 may be generated by functionalization, surface treatments, coatings or use of materials known in the art to limit, reduce, or reject the adhesion of collagen monomer or polymer units.

Single or multiple layers of collagen can be produced by regulating collagen self assembly conditions in the space bordered by the surfaces 820, 824 and inlet 810 and outlet 812. In preferred embodiments, the control of parameters such as flow rate and temperature are monitored and adjusted under computer system control, as described above.

In a preferred embodiment, the collagen monomer solution 804, as described above, enters the chamber through the inlet 810. The temperature of the surfaces 820 and 824 s regulated by the associated upper thermoelectric device 822 and lower thermoelectric device 826. In a preferred embodiment the upper thermoelectric device 822 is operated to cool the collagen rejecting surface 820 and the lower thermoelectric device 826 is operated to heat the collagen accepting surface 824.

Referring to FIG. 21B, in a preferred embodiment, layers of collagen in which the fibrils are aligned substantially orthogonal to the adjacent layers are formed by use of inlet 810 and corresponding outlet 812 alternating with use of inlet 814 and corresponding outlet 816. After the formation of a layer of aligned collagen fibrils in one direction, using inlet 810 and outlet 812, the micromanipulator 830 is used to increase the height 828 to provide space for an additional layer, and collagen monomer solution then flows from inlet 814 to outlet 816. Further iterations produces a multilaminar nanostructured artificial template. The input collagen monomer solution 804 is as described above. The outflow 808 is reduced in monomer concentration. Depending on flow rate and other parameters, the outflow 808 may be refluxed through the chamber 800 to improve the efficiency of use of monomer solution.

### EXAMPLE 8

Collagen constructs can be remodeled by alternating exposure to matrix metalloproteases (MMPs) and collagen monomers while maintained under load. The load can be a mechanical load, such as tensile stress, or a hydrostatic load caused by water absorption by retained moieties.

While not being held to a particular hypothesis, it is believed that collagen fibrils are “strain-stabilized”, i.e., the loading of the collagen fibril within the optimum in situ range of mechanical stress effectively shields the fibrils from degradation by proteolytic enzymes such as matrix metalloproteases (MMPs). As a self-assembling polymer, the degradation of collagen due to probability of enzymatic cleavage is in dynamic equilibrium with the addition of collagen monomers. Thus, when collagen fibrils are held in tension, the enzymatic action of Ps is reduced due to a reduced number of accessible MMP cleavage sites or due to a strain-induced reduction the MMP cleavage site accessibility. The enzymatic action of MMPs secreted by local activated fibroblasts only minimally affect collagen fibrils that are appropriately loaded, compared to unloaded neighboring collagen fibrils. This is consistent with several findings, including the loss of in vivo unloaded collagenous implants over time, the processes of collagen homeostasis, collagen removal and remodeling during wound repair, and collagen deposition during development.

It is believed, in general, that mechanical cues are critical to determining the differentiation state and activity of fibroblastic cells. In culture, the goal should be to provide mechanical conditions that are as near to in vivo conditions as possible. For the creation of a corneal construct, the relevant mechanical cues are collagen strain levels (subsequent to intraocular pressure) and trans matrix solute and solvent flux (also subsequent to intraocular pressure). In addition, there will also be a mechanical stress distribution across the construct due the pressure load as well. For other constructs such as fascia, tendon, ligament, the mechanical cues are the collagen strains due to mechanical loading of the ends of the construct. Whenever possible, the loading should be applied to reproduce the natural in vivo loading of the tissue that is to be regenerated. It should be understood that the load can be modulated as a collagen construct grows in order to maintain a constant effective stress.

If collagen fibrils are held in tension, the enzymatic action of MMPs is reduced, presumably due to MMP binding sites being less exposed to the enzyme. If collagen is loaded, the enzymatic action of local activated fibroblasts will affect it minimally compared to unloaded neighbor fibrils. Thus, collagen turnover can be directed simply by the difference in tension between the fibrils that are in use and those that are not. As observed in studies on loaded corneal strips described below, the fibrils that are unloaded tend to be removed by degradation by local MMPs. Thus, for a construct to be built in culture and maintained in vivo, the matrix must have appropriate loading to create an appropriate stabilizing strain on the couagen fibrils.

Nabeshima et al (Nabeshima, Y. et al., Uniaxial tension inhibits tendon collagen degradation by collagenase in vitro, J Orthopaed Res 1996 14 (1): 123-130) reported that rabbit patellar tendons loaded in tension resisted collagenase enzymatic breakdown compared to unloaded controls. Correction for diffusion demonstrated that tension did not limit the penetration of the collegenase into the tissue. Thus the mechanical effect of the tensile loading (which produced a strain of 4%) was sufficient to limit enzymatic breakdown of the matrix.

In addition to resisting enzymatic breakdown, tensile loads induce resistance to thermal breakdown. Bass et al (Bass, E. C., et al., Heat-Induced Changes in Annulus Fibrosus Biomechanics, J. Biomechanics, 2004, in press) reported that loading of porcine annulus fibrosus reduced the susceptibility of the collagen in the extracellular matrix to thermal degradation at 80° C. Loaded porcine annulus fibrosus exposed to heating showed no significant difference when compared to unloaded control annuli in subsequent denaturation experiments using modulated differential scanning calorimetry (MDSC).

In preferred embodiments, a construct comprising a load-bearing collagen, whether implanted or grown in culture, and containing collagen types I, II, III, V and XI or heterotypic fibrils, is strained by about 0.1 to about 20%, preferably about 0.5 to about 10%. Load bearing collagen is present in aligned connective tissues such as tendon, ligament, fascia, annulus fibrosus and cornea. For ligament and tendon constructs, clamps can be used to apply a mechanical load while cells generate additional matrix. For corneal constructs, a Ussing-type chamber (as shown in FIG. 19) can be used to apply a stress tangent to the plane of the construct

For cartilaginous constructs, where the collagenous fibrils comprise a cross-linked network, the collagen fibrils can be loaded by increasing the swelling pressure of the matrix with a space-filling component such as glycosaminoglycan, proteoglycan or other suitable polymer.

Once a manufactured construct is implanted in vivo, it must be loaded to avoid breakdown. Methods of providing a load to collagen-based implants in vivo include suturing in the implant into place under load in vivo (as in corneal transplant, or tendon/ligament transplant), providing a stiff framework to which the collagen is bound or other mechanism for creating tension in the fibrils of the construct.

One of the hallmarks of osteoarthritis (OA) is the loss of proteoglycans from the load bearing cartilage (Bank, R. A., et al., The increased swelling and instantaneous deformation of osteoarthritic cartilage is highly correlated with collagen degradation, Arthritis And Rheumatism 2000 43:2202-2210). Loss of proteoglycans reduces the swelling pressure of the cartilage which decreases the load necessary to “slacken” the typically taut collagen fibrils that comprise the collagen matrix in cartilage. Another cause of OA is an abnormally high loading of cartilage. Both conditions can lead to excessive compression of the cartilage matrix which induces “relaxation” of the collagen network (Basser, P. J., et al., Mechanical properties of the collagen network in human articular cartilage as measured by osmotic stress technique, Archives Of Biochemistry And Biophysics 1998 351:207-219). The unloading of the resident collagen fibrils could make them susceptible to enzymatic breakdown. As the amount of cartilage bearing the load decreases, the overall load adjacent to a defect would increase, making that region potentially susceptible to degradation. As more cartilage is resorbed, the local compressive load increases still further making more cartilage susceptible to cleavage.

In preferred embodiments, the invention provides a method of treating osteoarthritis comprising contacting the affected collagen fibrils with a retained hydrophilic moiety carrying a fixed charge density of about 0.1 to about 20 mEq/cm³.

Degeneration of the annulusfibrosus of the intervertebral disk (IVD) often follows degeneration or loss of the highly water sorbent proteoglycan, aggrecan, from the nucleus pulposus (Roughley, P. J., et al., The role of proteoglycans in aging, degeneration and repair of the intervertebral disc Biochem Soc T 2002 30:869-874). In the mechanical loading environment of the normal IVD, the nucleus pulposus (a soft, jelly-like collagenous/proteoglycan material), transmits compressive load to the annulusfibrosus (a tough, aligned, collagenous material) which carries the load in tension. Loss of the integrity of the nucleus pulposus results in a decline in the tensile load in the annulusfibrosus and ultimately the degeneration of the disk. Thus, any technique designed to restore the tensile loading environment to the annulusfibrosus following degeneration or loss of the annulus can serve to slow or reverse its degeneration.

It is thought that remodeling in bone (at least in subchondral bone) is partially due to tensile and bending forces (Eckstein, F., et al., Tension and bending, but not compression alone determine the functional adaptation of subchondral bone in incongruous joints Anal Embryol 1999 199: 85-97). Bone is a composite structure comprising a dense collagenous network that has been mineralized with calcium phosphate. As is the case with many degenerative processes associated with collagenous connective tissues, bone must be loaded to retain its mechanical properties. Merely compressive loads are not adequate to explain bone remodeling, some bending and tensile loading is required. It is the collagen in the bone matrix that is responsible for bearing the tensile loading of bone. Recently, more attention has been paid to the role of collagen turnover in osteoporosis patients (Mansell, J. P., et al., Increased metabolism of bone collagen in post-menopausal female osteoporotic femoral heads Int J Biochem Cell B 2003 35: 522-529).

In preferred embodiments, the present invention provides a method of remodeling a collagen construct by modulating the relative effects of 1) preferential enzymatic breakdown of collagen fibrils that are not adequately loaded (understrained) and 2) preferential incorporation of collagen monomers by collagen fibrils that are overloaded (overstrained) to reduce their strain values to within a set of limits specific to the type of collagen. In normal collagen homeostasis, underloaded collagen is constantly removed and overloaded collagen is reinforced. This mechanism is an intrinsic function of the collage/MMP system. The locally resident fibroblastic cells, which are capable of exerting influence by regulation of MMP or TIMP production, do not need to actively maintain the matrix when there is no pathology. The collagen/MMP system, if in balance, will maintain itself It is even capable of responding to new loading conditions with minimal cellular involvement (weightlessness or exercise).

The present invention provides a method for generating collagenous matrices of virtually any shape. In one embodiment, a random collagen gel may be produced from self-assembled collagen type I fibrils. The application of a uniaxial strain on the gel will place tension on a portion of the fibrils in the gel. The introduction of MMP specific for type I collagen should cleave fibrils not loaded adequately. Following the cleavage step, a dilute solution of activated monomer could be applied to the loaded matrix. Those fibrils that are overloaded should preferentially add monomer to bring their strain levels to within their control range. Repeating these steps should produce a collagen matrix, aligned with the load, which has fibrils of reasonably uniform diameter (i.e. a tendon or ligament). In another embodiment, following tensile loading of a random collagen get a solution containing low level of both monomer and MW could be added to produce the same effect. In another embodiment, the collagen gel could be loaded biaxially prior to MMP and monomer exposure to produce a tough collagen film with fibrils aligned with the loads. In another embodiment, the collagen gel could be loaded in any complex manner prior to MMP and monomer exposure to produce virtually any three-dimensional shape with aligned collagen fibrils gathered on the lines of loading.

In one embodiment, the invention provides a method for making an oriented collagenous structure, comprising the steps of providing a collagen construct, loading the collagen construct; contacting the collagen construct with a solution comprising at least one matrix metalloproteinase; and contacting the collagen construct with a solution comprising collagen monomers. In some embodiments, the method also includes the step of adjusting the load on the collagen construct In some embodiments, the method also includes the step of contacting the collagen construct with a population of cells. If desired, the step of contacting the collagen construct with a solution comprising at least one matrix metalloproteinase can be repeated one or more times. If desired, the step of contacting the collagen construct with a solution comprising collagen monomers can be repeated one or more times.

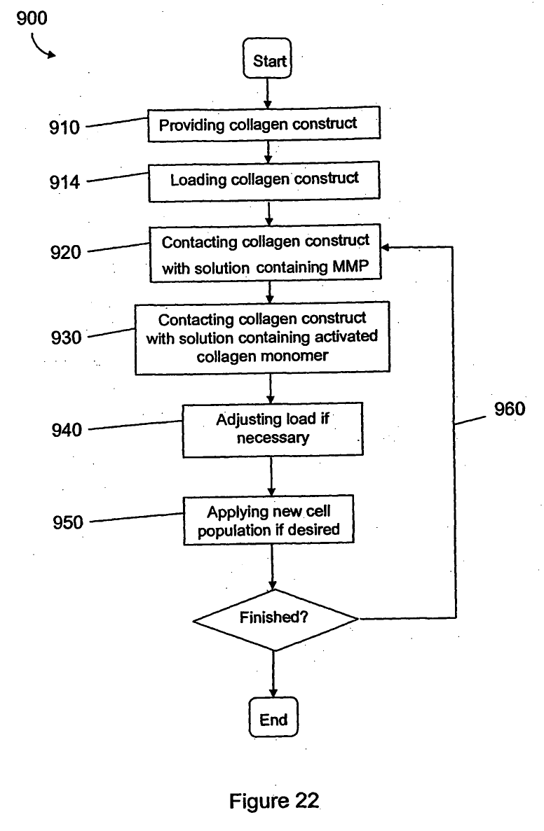

FIG. 22 is a flow chart describing method 900 for making an oriented collagenous structure in accordance with a preferred embodiment of the present invention. The method begins with step 910 of providing a collagen construct. The collagen construct can be an unoriented collagen gel, a tissue-derived collagen template or a nanostructured artificial template. Typically, the collagen construct comprises a collagen that is selected from the group consisting of Type I collagen, Type V collagen, and mixtures thereof In preferred embodiments, the collagen construct comprises Type I collagen. In other embodiments, the collagen construct comprises a mixture of Type I collagen and Type V collagen. Preferably, the construct comprises more Type I collagen than Type V collagen in one preferred embodiment, the construct comprises a mixture of about four parts Type I collagen to about one part Type V collagen. In some embodiments, the collagen construct can also include a collagen is selected from the group consisting of Type II collagen, Type m collagen, Type XI collagen, Type IV collagen, and mixtures thereof. The collagen fibrils can be homotypic or heterotypic.

Step 914, loading the collagen construct, can be accomplished either by application of external stress or production of hydrodynamic stress using retained internal hydrophilic moieties. A combination of both external stress and hydrodynamic stress using retained hydrophilic moieties can be used.

In preferred embodiments, a construct comprising a load-bearing collagen, whether implanted or grown in culture, and containing collagen types I, II, III, V and XI or heterotypic fibrils, is strained by about 0.1 to about 20%, preferably about 0.5 to about 10%. Load bearing collagen is present in aligned connective tissues such as tendon, ligament, fascia, annulus fibrosus and cornea. For ligament and tendon constructs, clamps can be used to apply a mechanical load while cells generate additional matrix. For corneal constructs, a Ussing-type chamber (as shown in FIG. 19) can be used to apply a stress tangent to the plane of the construct.

The collagen construct can be loaded with a static stress or a dynamic stress. Typically, the collagen construct is loaded with a stress sufficient to produce about 0.1% to about 20% strain, preferably about 0.5 to about 10% strain. Inpreferred embodiments, the collagen construct is loaded with a stress of about 0.01 to about 10 MPa. The applied stress can be oriented along a single axis (unaxial stress), or oriented along two axes (biaxial stress). In other embodiments, the collagen construct is loaded with a tangential stress. In further embodiments, the collagen construct is loaded with a three-dimensional stress.

In some embodiments, a planar collagen construct is placed between the half-chambers of an Ussing-style perfusion chamber and a pressure differential is applied between the half-chambers to apply a load on the collagen construct. A suitable perfusion chamber is illustrated schematically in FIG. 19. The perfusion chamber 600 has an upper portion 620 and a lower portion 640 that enclose the collagen construct, in this case a nanostructured artificial template 630. The upper portion 620 and a lower portion 640 are provided with respective perfusion ports 624 and 644. Fluids can be applied to the perfusion ports, and a pressure differential can apply stress to the nanostructured artificial template 630, causing it to deform into, preferably, a curved shape.

Alternatively, the collagen structure can be subjected to a stress by loading the collagen construct internally with at least one hydrophilic moiety that produces swelling pressure by the uptake of water. In such embodiments, the method includes the steps of providing a collagen construct; loading the collagen construct internally by adding at least one retained hydrophilic moiety that increases the swelling pressure of the collagen construct; contacting the collagen construct with a solution comprising at least one matrix metalloproteinase; and contacting the collagen construct with a solution comprising collagen monomers. Suitable hydrophilic moieties are “space-filling”, i.e. have the ability to imbibe fluid to generate a swelling pressure that is capable of tensioning the matrix in which they reside. In some embodiments, the method also includes the step of adjusting the load on the collagen construct.

In certain embodiments, the retained hydrophilic moiety is selected from the group consisting of glycosaminoglycans, proteoglycans and mixtures thereof. Where the retained hydrophilic moiety is a glycosaminoglycan, the glycosaminoglycan can be selected from the group consisting of hyaluran, chondroitin sulfate, dermatan sulfate, keratan sulfate, heparin, heparin sulfate, and mixtures thereof. Where the retained hydrophilic moiety is a proteoglycan, the proteoglycan can be selected from the group consisting of decorin, lumican, biglycan, keratocan, syndican, aggrecan, perlecan, asporin, fibromodulin, epiphycan, PG-Lb, dermatan sulfate proteoglycan-3, versican, mimecan and mixtures thereof. Alternatively, the retained hydrophilic moiety can be a polymer selected from the group consisting of polyvinyl alcohol, polyacrylic acid and mixtures thereof In other embodiments, the retained hydrophilic moiety is a biocompatible polymer with ionizable groups having a fixed charge density of about 0.01 to about 0.2 mEq/cm³.

The method 900 in accordance with the preferred embodiment includes the step 920 of contacting the collagen construct with a solution comprising at least one matrix metalloproteinase. The use of matrix metalloproteinases is known in the art. See, generally, Clark, L M., ed., Matrix Metalloproteinase Protocols, Humana Press, Totowa, N.J., 2001. The matrix metalloproteinase solution includes at least one includes a matrix metalloproteinase selected from the group consisting of MMP-1 (interstitial collagenase, EC 3.4.24.7), MMP-2 (gelatinase-A, EC 3A.24.24), MP-3 (stromelysin-1, transin, EC 3.4.24.17), MMP-7 (matrilysin-1, EC 3.4.24.23), MMP-8 (neutrophil collagenase, collagenase-2, EC 3.4.24.34), MMP-9 (gelatinase-B, EC 3.4.24.35), MMP-10, MP-1 (stromelysin-3), MMP-12 (metalloelastase macrophage elastase, EC 3.4.24.65), MMP-13 (collagenase-3, EC 3.4.24.-), MMP-18, recombinant catalytic domain fragments thereof and mixtures thereof Suitable matrix metalloproteinases are commercially available, e.g., from Sigma Aldrich Chemicals, St. Louis, Mo., or BIOMOL Research Laboratories, Inc., Plymouth Meeting, Pa. Matrix metalloproteinases can be used as a mixture of zymogen and active enzyme. However, greater control over the specific activity can be obtained by the use of recombinant catalytic domain fragments.

Recombinant catalytic domain fragments of matrix metalloproteinases are commercially available from BIOMOL Research Laboratories, Inc., Plymouth Meeting, Pa.: MMP-1 (19.9 kDa), MMP-2 (40 kDa), MMP-3 (19.5 kDa), MMP-7 (20.4 kDa), MMP-8 (20.3 kDa), MMP-9 (39 kDa), MMP-12 (20.3 kDa) and MMP-13 (20.4 kDa). Typically, suitable reaction conditions for each enzyme are those specified by the vendor. The particular matrix metalloproteinase selected depends on the specific type(s) of collagen that form the construct. If desired, the step of contacting the collagen construct with a solution comprising at least one matrix metalloproteinase can be repeated one or more times 960.

Step 930 is a step of contacting the collagen construct with a solution comprising collagen monomers. Suitable collagen monomer solutions are described in Examples 1, 2 and 5, above. In preferred embodiments, the temperature and flow of the monomer solution and the MMP solution are under the programmed control of a processor, such as a microprocessor, via pumps and heating-and cooling devices. Suitable heating and cooling devices are known in the art, including, without limitation, heat exchangers, resistive heaters and Peltier cells. The temperature of the monomer solution and the MMP solution are controlled to obtain the desired rates, respectively, of polymerization and the digestion of collagen fibrils not under load. Depending on the specific enzyme, the MMP solution can be maintained at the same or different temperature as the solution collagen monomer solution. If desired, the step of contacting the collagen construct with a solution comprising collagen monomers can be repeated one or more times 960.

In some preferred embodiments, the step 930 of contacting the collagen construct with a solution comprising at collagen monomers can precede the step 920 of contacting the collagen construct with a solution comprising at least one matrix metalloproteinase. Alternatively, steps 920 and 930 can be performed simultaneously. In situations where the pH, ionic conditions and additives required by the MMP enzyme are similar to the characteristics of the monomer solution required for polymerization, the monomers and the MMP may be included in the same solution.

The method 900 in accordance with the preferred embodiment includes the optional step 940 of adjusting the load. The applied stress can be adjusted to produce the desired amount of strain. In preferred embodiments, the load on the collagen construct is under programmed control. For example, the pressure differential across a construct in an Ussing chamber as described above can be monitored and controlled.

The method 900 also includes the optional step 950 of applying a new cell population to the collagen construct, as described in Example 6, above. The method 900 in accordance with the preferred embodiment includes the optional step 960 of repeating steps 920 and 930, and optionally 940 and 950.

In certain embodiments, the method includes the additional step of contacting the collagen construct with a solution comprising a proteoglycan selected from the group consisting of chondroitin sulfate, dermatan sulfate, keratan sulfate, decorin, lumican, biglycan, keratocan, syndican and mixtures thereof. In other embodiments, the method includes the step of contacting the collagen construct with a solution comprising a protein selected from the group consisting of collagen type IV, laminin, fibronectin, vinculin, an integrin moiety, and mixtures thereof.

As described above, in certain embodiments, the method includes the step of contacting the collagen construct with a solution comprising glycosaminoglycans, proteoglycans or mixtures thereof Where the solution comprises a glycosaminoglycan, the glycosaminoglycan can be selected from the group consisting of hyaluran, chondroitin sulfate, dermatan sulfate, keratan sulfate, heparin, heparin sulfate, and mixtures thereof Where the solution comprises a proteoglycan, the proteoglycan can be selected from the group consisting of decorin, lumican, biglycan, keratocan, syndican, aggrecan, perlecan asporin, fibromodulin, epiphycan, PG-Lb, dermatan sulfate proteoglycan-3, versican, mimecan and mixtures thereof In other embodiments, the method includes the step of contacting the collagen construct with a solution comprising a protein selected from the group consisting of collagen type IV, laminin, fibronectin, vinculin, an integrin moiety, and mixtures thereof.

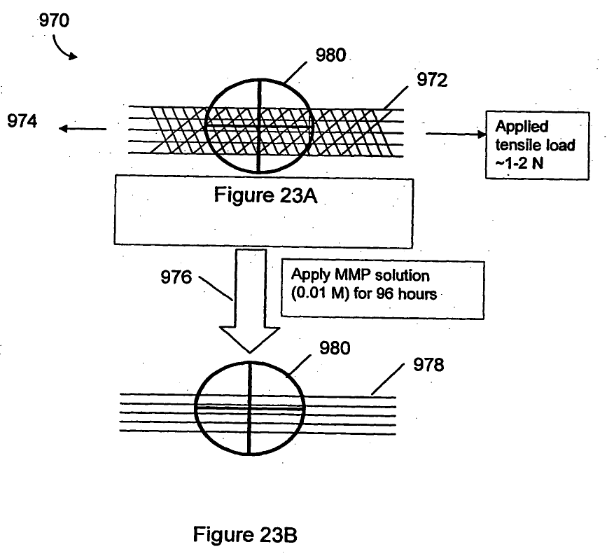

FIGS. 23A and 23B schematically illustrate the results of a study 970 in which collagen fibrils oriented parallel to an applied tensile load 978 were enzymatically selected from fibrils in the native corneal lamellae 972 as demonstrated by birefringence. Briefly, 2 mm×1 mm bovine corneal strips were cut to include central cornea at the center of the strip. The corneal strips were loaded with 1-2 N tension and subjected to collagenase at 37 degrees Celsius. The corneal strips were observed through crossed polarizers represented schematically 980 in FIGS. 23A and 23B. The corneal strips initially 972 (FIG. 23A) were observed to have unloaded fibrils oriented in several directions in addition to loaded fibrils aligned with the applied tensile stress, as a consequence of the plywood-like arrangement of the native corneal lamellae. After incubation with a solution containing 0.01 M collagenase for 96 hours 976, (FIG. 23B), only fibrils aligned with the applied tensile stress 978 were observed, indicating that the unloaded fibrils were preferentially degraded. This experiment incorporated the control fibris within the degraded matrix to assure that diffusion limitations could not explain the differential degradation. After 96 hours of exposure, aligning the major axis of either polarizer with the load direction demonstrated little or no birefringence while rotating the loaded sample axis to an angle 45° with respect to the axis of either polarizer demonstrated brightness indicating that fibrils aligned with the load remained intact.

The claims should not be read as limited to the described order or elements unless stated to that effect Therefore, all embodiments that come within the scope and spirit of the following claims and equivalents thereto are claimed as the invention.

1. A method of producing a templated extracellular matrix, comprising the steps of:
a) providing a substrate;
b) generating a nanostructured artificial template on the substrate using a shear flow; and
c) contacting the nanostructured artificial template with a population of cells activated for producing a templated extracellular matrix.

2-150. (canceled)

151. The method of claim 1, wherein the nanostructured artificial template is composed of aligned collagen.

152. The method of claim 1, wherein the substrate comprises a biocompatible textured surface, wherein the surface comprises aligned polymer, etched silicon, textured polymers, etched semi-conductor material, glass, or metals.

153. The method of claim 1, further comprising producing a structured connective tissue from the templated extracellular matrix, wherein the connective tissue is corneal stroma, ligament, tendon, fascia or annulus fibrosis.

154. The method of claim 1, wherein the step c) comprising contacting a layer of the nanostructured artificial template with the population of cells and maintaining the population of cells in a culture, thereby producing the templated extracellular matrix.

155. The method of claim 1, wherein the shear flow is generated by rotating the substrate to generate a thin film of a solution.

156. The method of claim 1, wherein the shear flow is generated by drawing the substrate out of a solution.

157. The method of claim 1, wherein the production of the templated extracellular matrix is controlled by at least one of the parameters of solution flow rate, solution viscosity, substrate rotational or pull velocity, solution and substrate temperature, solution pH, ambient humidity, solution chemistry, surface geometry, surface chemistry and surface wetting.

158. The method of claim 1, wherein a biomimetic corneal stroma is produced by the steps of:
a) providing a nanostructured artificial template;
b) contacting the nanostructured artificial template with a first population of eukaryotic cells;
c) maintaining the nanostructured artificial template and the first population of the cells in a culture to produce a templated extracellular matrix;
d) repeating the steps a) through c) to generate successive layers of templated extracellular matrices; and
e) stacking a plurality of the templated extracellular matrices oriented at any arbitrary angle with respect to one another to form a multilaminar templated extracellular matrix.

159. The method of claim 158, further comprising the steps of:
a) contacting a surface of the multilaminar templated extracellular matrix with a second population of cells; and
b) maintaining the multilaminar templated extracellular matrix and the second population of cells in a culture to produce a multilaminar templated extracellular matrix having at least one layer of the second population of cells on the surface.

160. The method of claim 158, further comprising the steps of:
a) contacting a second surface of the multilaminar templated extracellular matrix with a third population of cells; and
b) maintaining the multilaminar templated extracellular matrix and the third population of cells in a culture to produce a multilaminar templated extracellular matrix having at least one layer of the third population of the cells on the second surface.

161. A method of making a multilaminar nanostructured template comprising:
a) introducing a monomer solution from a first inlet into a confining region;
b) introducing a second monomer solution that forces the polymerization or association of the monomers through a second inlet;
c) confining the first and second solutions while polymerization or association proceeds;
d) modulating flow rate and/or confinement spacing to produce an aligned polymer layer; and
e) modulating flow direction to influence the adjacent layer alignment.

162. The method of claim 161, wherein the aligned polymers are formed by confining the solutions between two solid walls where one or both walls are polymer accepting or polymer rejecting.

163. The method of claim 161, wherein a single polymer filament is formed comprising the steps of:
a) providing a device having a channel, a first input opening, and a second input opening;
b) supplying a flow of the monomer solution to the first input opening to produce a first flow stream; and
c) supplying a flow of polymerizing agent to the second input opening to form a second flow stream;
wherein the first flow stream and the second flow stream join in an active zone at an angle greater than zero degrees to form a joined stream, and wherein the joined stream flows through the channel to the outflow opening, thereby producing a single polymer filament per channel.

164. The method of claim 161, further comprising providing a third flow stream to stop polymerization.

165. The method of claim 161, wherein the production of the multilaminar nanostructured template is controlled by at least one of the parameters of solution temperatures, wall confinement temperatures, solution chemistry, pH, surface chemistry, ambient temperature, humidity, flow rate of monomer solution, rate of polymerization agent, concentration of monomer, concentration of polymerizing agent, area of the interface in the active zone and relative flow rates.

166. The method of claim 161, wherein one or more channels are used to generate a multitude of parallel filaments.

167. A method for making a collagenous oriented structure comprising the steps of:
a) providing a collagen construct;
b) loading the collagen construct;
c) contacting the collagen construct with a solution comprising at least one matrix metalloproteinase; and
d) contacting the collagen construct with a solution comprising collagen monomers.

168. The method of claim 167, further comprising a step of adjusting the load on the collagen construct.

169. The method of claim 167, further comprising a step of contacting the collagen construct with a solution, wherein the solution comprises at least one compound selected from the group consisting of hyaluronan, chondroitin sulfate, dermatan sulfate, aggrecan, keratan sulfate, decorin, lumican, biglycan, keratocan, syndican, collagen type Pt, laminin, fibronectin, vinculin, an integrin moiety, hyaluronan, chondroitin sulfate, dermatan sulfate, keratan sulfate, heparin, heparin sulfate, and mixtures thereof.
